(19) United States
(12) Patent Application Publication
(10) Pub. No.: US 2007/0032521 A1
(43) Pub. Date: Feb. 8, 2007

(54) Use of c-kit inhibitors for treating type II diabetes

(75) Inventors: Alain Moussy, Paris (FR); Jean-Pierre Kinet, Lexington, MA (US)

Correspondence Address: FOLEY AND LARDNER LLP;SUITE 500, 3000 K STREET NW, WASHINGTON, DC, 20007, US

(73) Assignee: AB Science

(21) Appl. No.: 10/567,557

(22) PCT Filed: Aug. 16, 2004
(86) PCT No.: PCT/IB04/02934

§ 371 Date: May 11, 2006

Related U.S. Application Data
(60) Provisional application No. 60/495,088, filed on Aug. 15, 2003.

(51) Int. Cl. A61K 31/47

(52) U.S. Cl. 514/310

(57) ABSTRACT

The present invention relates to a method for treating type II diabetes, comprising administering a compound capable of depleting mast cells to a human in need of such treatment. Such compounds can be chosen from non-toxic, selective and potent c-kit inhibitors.

The present invention relates to a method for treating type II diabetes, obesity and related disorders comprising administering a compound capable of depleting mast cells or a compound inhibiting mast cells degranulation, to a human in need of such treatment Such compounds can be chosen from c-kit inhibitors and more particularly non-toxic, selective and potent c-kit inhibitors. Preferably, said inhibitor is unable to promote death of IL-3 dependent cells cultured in presence of IL-3.

Non-insulin-dependent diabetes mellitus (NDDM), also known as type II diabetes, is defined as a chronic disease appearing when the insulin turns out to be inefficient in promoting glucose uptake by cells, which results in increased levels of glucose in the blood. This disease affects about 100 million people world-wide, 75% of which are obese at the time of diagnosis.

Diminution in the ability of the cells to respond adequately to insulin is often referred as insulin resistance. Excessive weight and lack of physical activity are regarded as being responsible for inducing insulin resistance. Over many years, the failure of the glucose uptake regulation leads to the development of Type II diabetes and the blood glucose level needs to be regulated with medicinal products. Ultimately, unregulated blood glucose level is responsible for blood vessels, kidney and eye damages, as well as cardiovascular diseases. This tissue damages contribute to mortality in diabetics.

Hypoglycemic agents such as sulfonylureas work by triggering the pancreas to make more insulin, which lower blood glucose. The side effects of sulfonylureas include hypoglycemia, renal and hepatic disease, gastrointestinal disturbances, increased cardiovascular mortality, dermatological reactions, drowsiness and headache. Biguanides lower blood glucose levels by reducing intestinal glucose absorption and hepatic glucose, but not by stimulating insulin secretion. The major side effects of biguanidine are lactic acidosis and increased cardiovascular mortality. Alpha-glucosidase inhibitors decrease the absorption of carbohydrates from the digestive tract, thereby lowering the after-meal glucose level, but gastrointestinal side effects and hypoglycemia are observed. Thiazolidinediones, such as rosiglitazone are PPARgamma agonists and increase the cell's sensitivity to insulin. However, they may be responsible for water retention, liver diseases, cardiovascular diseases, red blood cell abnormalities, and headache.

Because treatment of Type II diabetes requires long term administration of compounds lowering blood glucose level, there is still a great need for improved and safer methods.

In connection with the present invention, we have unexpectedly discovered that c-kit inhibitors lower the level of glucose, cholesterol, triglycerides and non esterified fatty acids in blood.

In addition, these inhibitors do not affect significantly the level of insulin contrary to compounds of the thiazolidinedione family.

This observation is surprising and we can only speculate at this time of the mechanism of action of c-kit inhibitors. We know that c-kit is of crucial importance for activation of mast cells. Following mast cells activation, released granules liberate various factors which could directly or indirectly participate in the regulation of different metabolites uptake and processing by the cells. Among such factors, we can cite a cocktail of different proteases, lipid-derived mediators (prostaglandins, thromboxanes and leucotrienes) and various cytokines (IL-1, IL-2, IL-3, IL-4, IL-5, IL-6, IL-8, TNF-α, GM-CSF, MIP-1a, MIP-1b, MIP-2 and IFN-γ). In Lyon C J, et al, Proc Nutr Soc 2001 Aug; 60(3):329-39 is it mentioned that adipose tissue is a dynamic endocrine organ that secretes a number of factors that are increasingly recognized to contribute to systemic and vascular inflammation.

The major secretory compartment of adipose tissue consists of adipocytes, fibroblasts, and mast cells. These cells, using endocrine, paracrine and autocrine pathways, secrete multiple bioactive molecules, conceptualized as “adipokines”.

Here, based on our observation that c-kit inhibitors works in lowering notably blood glucose, we postulate that mast cells regulate, directly or indirectly, a number of the processes that contribute to the development of atherosclerosis, including hypercholesterolemia, hypergycemia, hypertension, endothelial dysfunction, insulin resistance, and vascular remodeling. But, as this point, other mechanisms may not be ruled out.

A new route for treating type II diabetes, obesity and related disorders is provided, which consists of administering c-kit inhibitors to patients.

### DESCRIPTION

The present invention relates to a method for treating type II diabetes, obesity and related disorders comprising administering a compound capable of depleting mast cells or blocking mast cells degranulation to a human in need of such treatment.

Said method for treating type II diabetes can comprise administering a c-kit inhibitor to a human in need of such treatment. Alternatively, it may also consist of administering an antihistamine compound or a compound that blocks mast cells exocytosis such as the Rigel's pharmaceuticals R112.

Preferred compounds are c-kit inhibitor, more particularly a non-toxic, selective and potent c-kit inhibitor. Such inhibitors can be selected from the group consisting of 2-(3-amino)arylamino-4-aryl-thiazoles, pyrimidine derivatives, pyrrolopyrimidine derivatives, quinazoline derivatives, quinoxaline derivatives, pyrazoles derivatives, bis monocyclic, bicyclic or heterocyclic aryl compounds, vinylene-azaindole derivatives and pyridyl-quinolones derivatives, styryl compounds, styryl-substituted pyridyl compounds, seleoindoles, selenides, tricyclic polyhydroxylic compounds and benzylphosphonic acid compounds.

Among preferred compounds, it is of interest to focus on pyrimidine derivatives such as N-phenyl-2-pyrimidine-amine derivatives (U.S. Pat. No. 5,521,184 and WO 99/03854), indolinone derivatives and pyrrol-substituted indolinones (U.S. Pat. No. 5,792,783, EP 934 931, U.S. Pat. Nos. 5,834,504), 5,883,116, 5,883,113, 5,886,020, WO 96/40116 and WO 00/38519), as well as bis monocyclic, bicyclic aryl and heteroaryl compounds (EP 584 222, U.S. Pat. No. 5,656,643 and WO 92/20642), quinazoline derivatives (EP 602 851, EP 520 722, U.S. Pat. Nos. 3,772,295 and 4,343,940), 4-amino-substituted quinazolines (U.S. Pat No. 3,470,182), 4-thienyl-2-(1H)-quinazolones, 6,7-dialkoxyquinazolines (U.S. Pat. No. 3,800,039), aryl and heteroaryl quinazoline (U.S. Pat. Nos. 5,721,237, 5,714,493, 5,710,158 and WO 95/15758), 4-anilinoquinazoline compounds (U.S. Pat. No. 4,464,375), and 4-thienyl-2-(1H)-quinazolones (U.S. Pat. No. 3,551,427).

So, preferably, the invention relates to a method for treating type II diabetes comprising administering a non toxic, potent and selective c-kit inhibitor is a pyrimidine derivatives, more particularly N-phenyl-2-pyrimidine-amine derivatives of formula I: 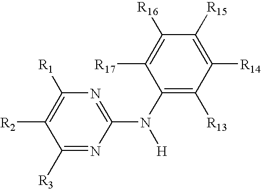
wherein the R1, R2, R3, R13 to R17 groups have the meanings depicted in EP 564 409 B1, incorporated herein in the description.

Preferably, the N-phenyl-2-pyrimidine-amine derivative is selected from the compounds corresponding to formula II: 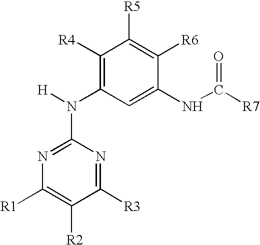
Wherein R1, R2 and R3 are independently chosen from H, F, Cl, Br, I, a C1-C5 alkyl or a cyclic or heterocyclic group, especially a pyridyl group;
R4, R5 and R6 are independently chosen from H, F, Cl, Br, I, a C1-C5 alkyl, especially a methyl group;
and R7 is a phenyl group bearing at least one substituent, which in turn possesses at least one basic site, such as an amino function.

Preferably, R7 is the following group:

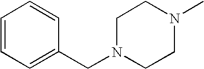

Among these compounds, the preferred are defined as follows:
R1 is a heterocyclic group, especially a pyridyl group,
R2 and R3 are H,
R4 is a C1-C3 alkyl, especially a methyl group,
R5 and R6 are H,
and R7 is a phenyl group bearing at least one substituent, which in turn possesses at least one basic site, such as an amino function, for example the group: 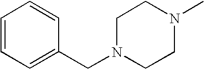
   Therefore, in a preferred embodiment, the invention relates to a method for treating type II diabetes comprising the administration of an effective amount of the compound known in the art as CGP57148B:
4-(4-mehylpipérazine-1-ylméthyl)-N-[4-méthyl-3-(4-pyridine-3-yl)pyrimidine-2 ylamino)phényl]-benzamide corresponding to the following formula: 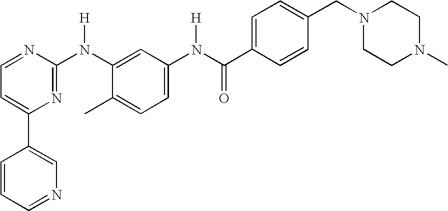
   The preparation of this compound is described in example 21 of EP 564 409 and the β-form, which is particularly useful is described in WO 99/03854.
   Alternatively, the c-kit inhibitor can be selected from:
   indolinone derivatives, more particularly pyrrol-substituted indolinones,
   monocyclic, bicyclic aryl and heteroaryl compounds, quinazoline derivatives,
      and quinaxolines, such as 2-phényl-quinaxoline derivatives, for example 2-phenyl-6,7-dimethoxy quinaxoline.

In a preferred another preferred embodiment, the invention contemplated the method mentioned above, wherein said c-kit inhibitor is selected from 2-(3-amino)arylamino-4-aryl-thiazoles such as those chosen from formula III for which the applicant filed U.S. Ser. No. 60/400064: 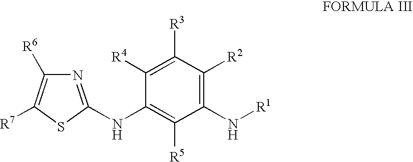
and wherein R¹ is:
a) a linear or branched alkyl group containing from 1 to 10 carbon atoms optionally substituted with at least one heteroatom, notably a halogen selected from I, Cl, Br and F, and/or bearing a pendant basic nitrogen functionality;
b) an aryl or heteroaryl group optionally substituted by an alkyl or aryl group optionally substituted with a heteroatom, notably a halogen selected from I, Cl, Br and F or bearing a pendant basic nitrogen functionality;
c) a —CO—NH—R, —CO—R, —CO—OR or a —CO—NRR′ group, wherein R and R′ are independently chosen from H or an aryl, heteroaryl, alkyl and cycloalkyl group optionally substituted with at least one heteroatom, notably a halogen selected from I, Cl, Br and F, and/or bearing a pendant basic nitrogen functionality;
R² is hydrogen, halogen or a linear or branched alkyl group containing from 1 to 10 carbon atoms, trifluoromethyl or alkoxy;
R³ is hydrogen, halogen or a linear or branched alkyl group containing from 1 to 10 carbon atoms, trifluoromethyl or alkoxy,
R⁴ is hydrogen, halogen or a linear or branched alkyl group containing from 1 to 10 carbon atoms, trifluoromethyl or alkoxy;
R⁵ is hydrogen, halogen or a linear or branched alkyl group containing from 1 to 10 carbon atoms, trifluoromethyl or alkoxy;
R⁶ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy,
iv) H, a halogen selected from I, F, Cl or Br, NH2, NO2 or SO2-R, wherein R is a linear or branched alkyl goup containing one or more group such as 1 to 10 carbon atoms, and optionally substituted with at least one heteroatom, notably a halogen selected from I, Cl, Br and F, and/or bearing a pendant basic nitrogen functionality;
   and R⁷ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.
iv) H, a halogen selected from I, F, Cl or Br; NH2, NO2 or SO2-R, wherein R is a linear or branched alkyl goup containing one or more group such as 1 to 10 carbon atoms, and optionally substituted with at least one heteroatom, notably a halogen selected from I, Cl, Br and F, and/or bearing a pendant basic nitrogen functionality.

In another preferred embodiment, when R¹ has the meaning depicted in c) above, the invention is directed to compounds of the following formula: 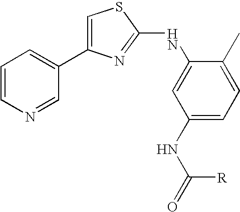
wherein R is H or an organic group that can be selected for example from a linear or branched alkyl group containing from 1 to 10 carbon atoms optionally substituted with at least one heteroatom or bearing a pendant basic nitrogen functionality; a cycloalkyl, an aryl or heteroaryl group optionally substituted by an alkyl, a cycloalkyl, an aryl or heteroaryl group optionally substituted with a heteroatom, notably a halogen selected from I, Cl, Br and F and/or bearing a pendant basic nitrogen functionality.

Among the particular compounds in which RI has the meaning as depicted in c) above, the invention is directed to amideaniline compounds of the following formula: 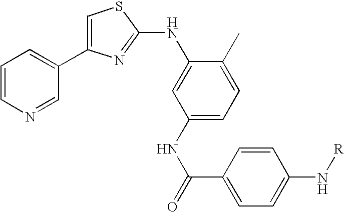
wherein R is H or an organic group that can be selected for example from a linear or branched alkyl group containing from 1 to 10 carbon atoms optionally substituted with at least one heteroatom or bearing a pendant basic nitrogen functionality; a cycloalkyl, an aryl or heteroaryl group optionally substituted with a heteroatom, notably a halogen selected from I, Cl, Br and F and/or bearing a pendant basic nitrogen functionality; or a a cycloalkyl, an aryl or heteroaryl group optionally substituted with a cycloalkyl, an aryl or heteroaryl group optionally substituted with a heteroatom, notably a halogen selected from I, Cl, Br and F and/or bearing a pendant basic nitrogen functionality;
a —SO2-R group wherein R is an alkyl, cycloalkyl, aryl or heteroaryl optionally substituted with an heteroatom, notably a halogen selected from I, Cl, Br and F and/or bearing a pendant basic nitrogen functionality; or a —CO—R or a —CO—NRR′ group, wherein R and R′ are independently chosen from H, an alkyl, a cycloalkyl, an aryl or heteroaryl group optionally substituted with at least one heteroatom, notably selected from I, Cl, Br and F, and/or bearing a pendant basic nitrogen functionality.

Among the particular compounds in which RI has the meaning as depicted in c) above, the invention is directed to amide-benzylamine compounds of the following formula: 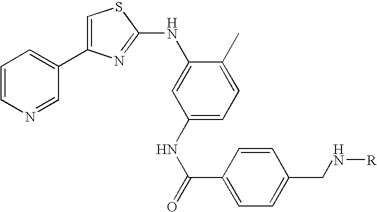
wherein R is H or an organic group that can be selected for example from a linear or branched alkyl group containing from 1 to 10 carbon atoms optionally substituted with at least one heteroatom, notably a halogen selected from I, Cl, Br and F, and/or bearing a pendant basic nitrogen functionality, a cycloalkyl, aryl or heteroaryl group optionally substituted with an heteroatom, notably a halogen selected from I, Cl, Br and F or bearing a pendant basic nitrogen functionality; or an alkyl, cycloalkyl, aryl or heteroaryl group substituted by a alkyl, cycloalkyl, aryl or heteroaryl group optionally substituted with a heteroatom, notably a halogen selected from I, Cl, Br and F or bearing a pendant basic nitrogen functionality;
a —SO2-R group wherein R is an alkyl, cycloalkyl, aryl or heteroaryl group optionally substituted with an heteroatom, notably a halogen selected from I, Cl, Br and F or bearing a pendant basic nitrogen functionality; or a —CO—R or a —O—NRR′ group, wherein R and R′ are independently chosen from H or an aryl heteroaryl, alkyl and cycloalkyl group optionally substituted with at least one heteroatom and/or bearing a pendant basic nitrogen functionality.

Among the particular compounds in which R1 has the meaning as depicted in c) above, the invention is directed to amide-phenol compounds of the following formula: 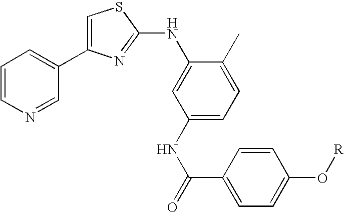
wherein R is H or an organic group that can be selected for example from a linear or branched alkyl group containing from 1 to 10 carbon atoms optionally substituted with at least one heteroatom, notably a halogen selected from I, Cl, Br and F, and/or bearing a pendant basic nitrogen functionality;
a cycloalkyl, aryl or heteroaryl group optionally substituted with a heteroatom, notably a halogen selected from I, Cl, Br and F and/or bearing a pendant basic nitrogen functionality, or an alkyl, cycloalkyl, aryl or heteroaryl group substituted by a alkyl, cycloalkyl, aryl or heteroaryl group optionally substituted with a heteroatom, notably a halogen selected from I, Cl, Br and F and/or bearing a pendant basic nitrogen functionality;
a -SO2-R group wherein R is an alkyl, cycloalkyl, aryl or heteroaryl group optionally substituted with an heteroatom, notably a halogen selected from I, Cl, Br and F and/or bearing a pendant basic nitrogen functionality; or a —CO—R or a —CO—NRR′ group, wherein R and R′ are independently chosen from H or an aryl, heteroaryl, alkyl and cycloalkyl group optionally substituted with at least one heteroatom and/or bearing a pendant basic nitrogen functionality.

Among the particular compounds in which R1 has the meaning as depicted in c) above, the invention is directed to urea compounds of the following formula: 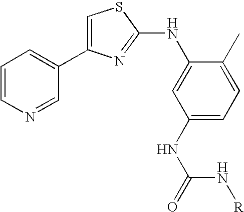
wherein R is H or an organic group that can be selected for example from a linear or branched alkyl group containing from 1 to 10 carbon atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality.

Among the particular compounds in which R1has the meaning as depicted in a) and b) above, the invention is directed to N-Aminoalkyl-N-thiazol-2-yl-benzene-1,3-diamine compounds of the following formula: 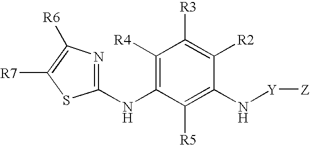
wherein Y is a linear or branched alkyl group containing from 1 to 10 carbon atoms;
wherein Z represents an aryl or heteroaryl group, optionally substituted at one or more ring position with any permutation of the following groups:
   a halogen such as F, Cl, Br, I;
   a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
   an O—R, where R is a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
   an NRaRb, where Ra and Rb represents a hydrogen, or a linear or branched alkyl group containing from 1 to 10 carbon atoms atoms optionally substituted with at least one heteroatom (for example a halogen) and/or bearing a pendant basic nitrogen functionality or a cycle; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
   a COOR, where R is a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
   a CONRaRb, where Ra and Rb are a hydrogen or a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality, or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
   an NHCOR, where R is a linear or branched alkyl group containing from 1 to 10 carbon atoms atoms optionally substituted with at least one heteroatom (for example a halogen) and/or bearing a pendant basic nitrogen functionality, a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
   an NHCOOR, where R is a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
   an NHCONRaRb, where Ra and Rb are a hydrogen or a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality, or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
   an OS0₂R, where R is a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
   an NMaOSO₂Rb, where Ra and Rb are a linear or branched alkyl group containing from 1 to 10 carbon atoms atoms optionally substituted with at least one heteroatom (for example a halogen) and/or bearing a pendant basic nitrogen functionality; Ra can also be a hydrogen; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an allyl, a cycloalkyl, an aryl or heteroaryl group optionally substituted with an heteroatom, notably a halogen selected from I, Cl, Br and F, and/or bearing a pendant basic nitrogen functionality;
R² is hydrogen, halogen or a linear or branched alkyl group containing from 1 to 10 carbon atoms, trifluoromethyl or alkoxy;
R³ is hydrogen, halogen or a linear or branched alkyl group containing from 1 to 10 carbon atoms, trifluoromethyl or alkoxy;
R⁴ is hydrogen, halogen or a linear or branched alkyl group containing from 1 to 10 carbon atoms, trifluoromethyl or alkoxy;
R⁵ is hydrogen, halogen or a linear or branched alkyl group containing from 1 to 10 carbon atoms, trifluoromethyl or alkoxy;
R⁶ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.
iv) H, a halogen selected from I, F, Cl or Br; NH2, NO2 or SO2-R, wherein R is a linear or branched alkyl goup containing one or more group such as 1 to 10 carbon atoms, and optionally substituted with at least one heteroatom, notably a halogen selected from I, Cl, Br and F, and/or bearing a pendant basic nitrogen functionality;
and R⁷ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.
iv) H, an halogen selected from I, F, Cl or Br; NH2, NO2 or SO2-R, wherein R is a linear or branched alkyl goup containing one or more group such as 1 to 10 carbon atoms, and optionally substituted with at least one heteroatom, notably a halogen selected from I, Cl, Br and F, and/or bearing a pendant basic nitrogen functionality.

An example of preferred compounds of the above formula is depicted below:

### 001: 4-{[4-Methyl-3-(4-pyridin-3-yl-thiazol-2-ylamino)-phenylamino]-methyl}-benzoic acid methyl ester

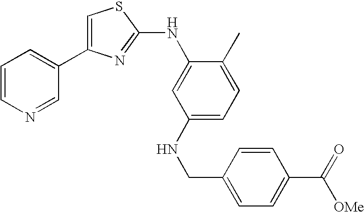

Among the compounds of formula I, the invention is particularly embodied by the compounds of the following formula IV: 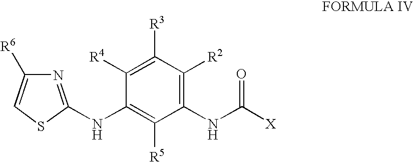
wherein X is R or NRR′ and wherein R and R′ are independently chosen from H, an aryl, a heteroaryl, an alkyl, or a cycloalkyl group optionally substituted with at least one heteroatom, such as for example a halogen chosen from F, I, Cl and Br and optionally bearing a pendant basic nitrogen functionality; or an aryl, a heteroaryl, an alkyl or a cycloalkyl group substituted with an aryl, a heteroaryl, an alkyl or a cycloalkyl group optionally substituted with at least one heteroatom, such as for example a halogen chosen from F, I, Cl and Br and optionally bearing a pendant basic nitrogen functionality,
R² is hydrogen, halogen or a linear or branched alkyl group containing from 1 to 10 carbon atoms, trifluoromethyl or alkoxy;
R³ is hydrogen, halogen or a linear or branched alkyl group containing from 1 to 10 carbon atoms, trifluoromethyl or alkoxy;
R⁴ is hydrogen, halogen or a linear or branched alkyl group containing from 1 to 10 carbon atoms, trifluoromethyl or alkoxy;
R⁵ is hydrogen, halogen or a linear or branched alkyl group containing from 1 to 10 carbon atoms, trifluoromethyl or alkoxy;
R⁶ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.
iv) H, a halogen selected from I, F, Cl or Br, NH2, NO2 or SO2-R, wherein R is a linear or branched alkyl goup containing one or more group such as 1 to 10 carbon atoms, and optionally substituted with at least one heteroatom, notably a halogen selected from I, Cl, Br and F, and/or bearing a pendant basic nitrogen functionality.

In another alternative, substituent R6, which in the formula II is connected to position 4 of the thiazole ring, may instead occupy position 5 of the thiazole ring.

Among the preferred compounds corresponding formula IV, the invention is directed to compounds in which X is a substituted alkyl, aryl or heteroaryl group bearing a pendant basic nitrogen functionality represented for example by the structures a to f shown below, wherein the wavy line corresponds to the point of attachment to core structure of formula IV:

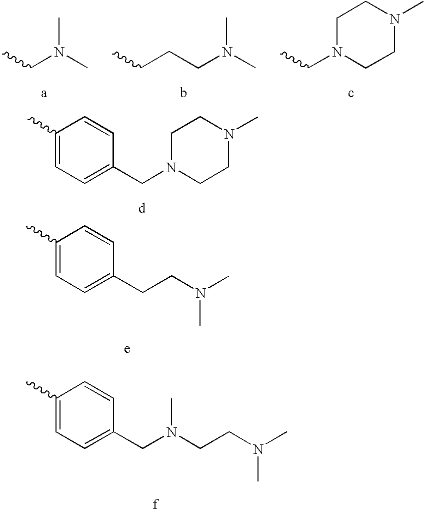

Among group a to f, X (see formula II) is preferentially group d.

Furthermore, among the preferred compounds of formula III or IV, the invention concerns the compounds in which R² and R³ are hydrogen. Preferentially, R⁴ is a methyl group and R⁵ is H. In addition, R⁶ is preferentially a 3-pyridyl group (cf. structure g below), or a 4-pyridyl group (cf. structure h below). The wavy line in structure g and h correspond to the point of attachment to the core structure of formula III or IV.

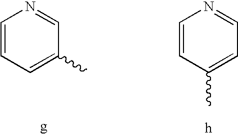

Thus, the invention contemplates:
1—A compound of formula IV as depicted above, wherein X is group d and R⁶ is a 3-pyridyl group.
2—A compound of formula IV as depicted above, wherein X is group d and R⁴ is a methyl group.
3—A compound of formula III or IV as depicted above, wherein R¹ is group d and R² is H.
4—A compound of formula III or IV as depicted above, wherein R¹ is group d and R³ is H.
5—A compound of formula III or IV as depicted above, wherein R¹ is group d and R² and/or R³ and/or R⁵ is H.
6—A compound of formula III or IV as depicted above, wherein R⁶ is a 3-pyridyl group and R³ is a methyl group.
7—A compound of formula III or IV as depicted above, wherein R⁶ is a 3-pyridyl group and R² is H.
8—A compound of formula III or IV as depicted above, wherein R² and/or R³ and/or R⁵ is H and R⁴ is a methyl group.
9—A compound of formula III or IV as depicted above wherein R² and/or R³ and/or R⁵ is H, R⁴ is a methyl group and R⁶ is a 3-pyridyl group.

Among the compounds of formula IV, the invention is particularly embodied by the compounds wherein R2, R3, R5 are hydrogen, corresponding to the following formula IV-1: 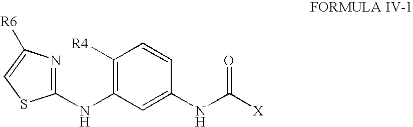
wherein X is R or NRR′ and wherein R and R′ are independently chosen from H or an organic group that can be selected for example from a linear or branched alkyl group containing from 1 to 10 carbon atoms optionally substituted with at least one heteroatom or bearing a pendant basic nitrogen functionality; a cycloalkyl, an aryl or heteroaryl group optionally substituted with an heteroatom, notably a halogen selected from I, Cl, Br and F or bearing a pendant basic nitrogen functionality, or a a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;
a —SO2-R group wherein R is an alkyl, cycloalkyl, aryl or heteroaryl optionally substituted with a heteroatom, notably a halogen selected from I, Cl, Br and F or bearing a pendant basic nitrogen functionality; or a —CO—R or a —CO—NRR′ group, wherein R and R′ are independently chosen from H, an alkyl, a cycloalkyl, an aryl or heteroaryl group optionally substituted with at least one heteroatom, notably selected from I, Cl, Br and F, and/or bearing a pendant basic nitrogen functionality.
R⁴ is hydrogen, halogen or a linear or branched alkyl group containing from 1 to 10 carbon atoms, trifluoromethyl or alkoxy;
R⁶ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.
iv) H, a halogen selected from I, F, Cl or Br; NH2, NO2 or SO2-R, wherein R is a linear or branched alkyl goup containing one or more group such as 1 to 10 carbon atoms, and optionally substituted with at least one heteroatom, notably a halogen selected from I, Cl, Br and F, and/or bearing a pendant basic nitrogen functionality.

In another alternative, substituent R6, which in the formula II is connected to position 4 of the thiazole ring, may instead occupy position 5 of the thiazole ring.

### EXAMPLES

### 002: 2-(2-methyl-5-amino)phenyl-4-(3-pyridyl)-thiazole

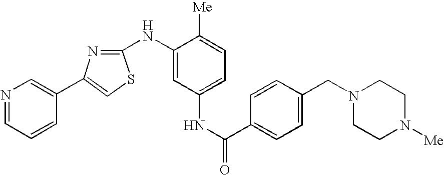

### 003: 4-(4-Methyl-piperazin-l-ylmethyl)-N-[3-(4-pyridin-3-yl-thiazol-2-ylamino)-phenyl]-benzamide

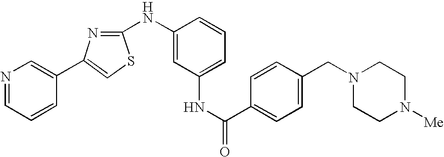

### 004: N-[4-Methyl-3-(4-phenyl-thiazol-2-ylamino)-phenyl]-4-(4-methyl-piperazin-1-ylmethyl)-benzamide

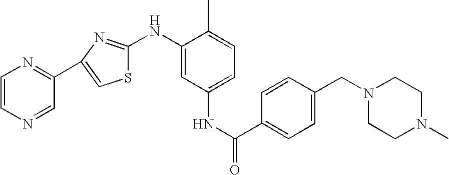

### 005: N-[3-([2,4]Bithiazolyl-2′-ylamino)-4-methyl-phenyl]-4-(4-methyl-piperazin-1-ylmethyl)-benzamide

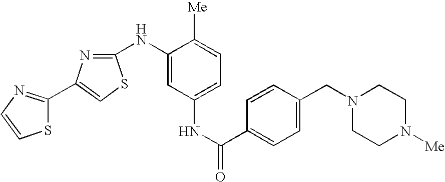

### 006: 4-(4-Methyl-piperazin-1-ylmethyl)-N-[4-methyl-3-(4-pyrazin-2-yl-thiazol-2-ylamino)-phenyl]-benzamide

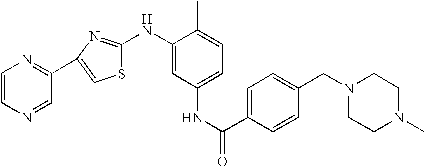

### 007: 2-[5-(3-Iodo-benzoylamino)-2-methyl-phenylamino]-thiazole-4-carboxylic acid ethyl ester

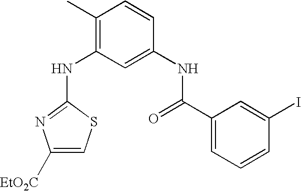

### 008: 2-{2-Methyl-5-[4-(4-methyl-piperazin-1-ylmethyl)-benzoylamino]-phenylamino}-thiazole-4-carboxylic acid ethyl ester

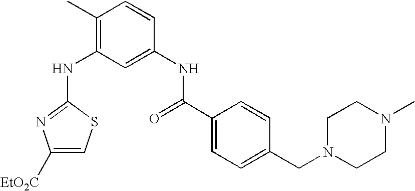

### 027: 2-(2-chloro-5-amino)phenyl-4-(3-pyridyl)-thiazole

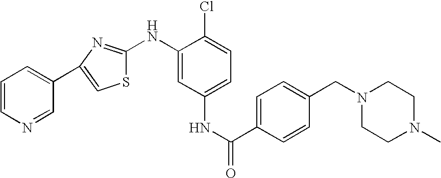

### 128: 3-Bromo-N-{3-[4-(4-chloro-phenyl)-5-methyl-thiazol-2-ylamino]-4-methyl-phenyl}-benzamide

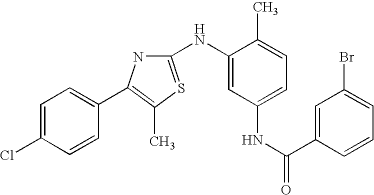

### 129: {3-[4-(4-Chloro-phenyl)-5-methyl-thiazol-2-ylamino]-4-methyl-phenyl}-carbamic acid isobutyl ester

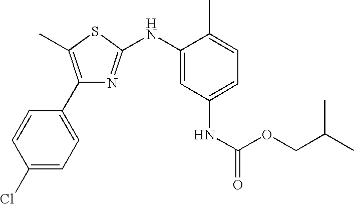

### 130: 2-[5-(3-Bromo-benzoylamino)-2-methyl-phenylamino]-5-(4-chloro-phenyl)-thiazole-4-carboxylic acid ethyl ester

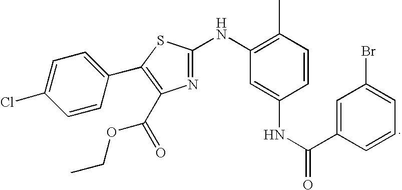

### 131: 2-[5-(3-Bromo-benzoylamino)-2-methyl-phenylamino]-5-(4-chloro-phenyl)-thiazole-4-carboxylic acid (2-dimethylamino-ethyl)-amide

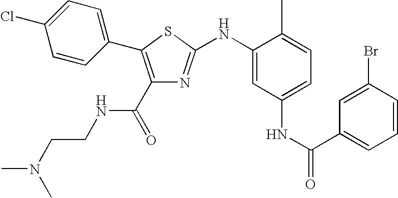

### 110: N-{3-[4-(4-Methoxy-phenyl)-thiazol-2-ylamino]-4-methyl-phenyl}-4-(4methyl-piperazin-1 ylmethyl)-benzamide

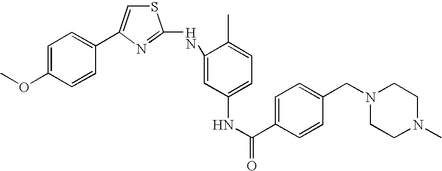

### 116: 4-(4-Methyl-piperazin-1-ylmethyl)-N-{4-methyl-3-[4-(3-trifluoromethyl-phenyl)-thiazol-2-ylamino]-phenyl}-benzamide

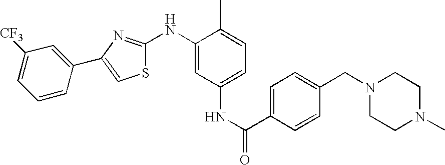

### 117: N-{4-Methyl-3-[4(3-nitro-phenyl)-thiazol-2-ylamino]-phenyl}-4-(4-methyl-piperazin-1-ylmethyl)-benzamide

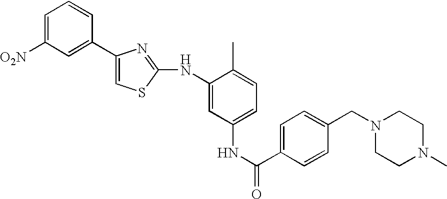

### 124: N-{3-[4-(2,5-Dimethyl-phenyl)thiazol-2-ylamino]-4-methyl-phenyl}-4-(4-methyl-piperazin-1-ylmethyl)-benzamide

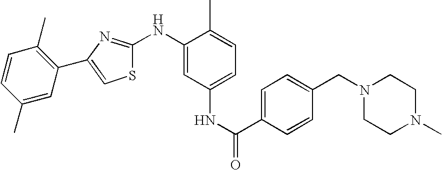

### 108: N-{3-[4-(4-Chloro-phenyl)-thiazol-2-ylamino]-4-methyl-phenyl}-4-(4-methyl-piperazin-1-ylmethyl)-benzamide

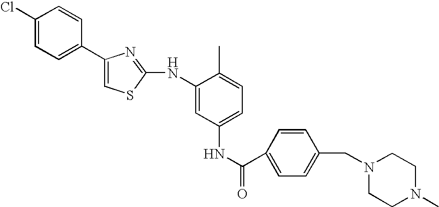

### 113: N-{3-[4-(3-Methoxy-phenyl)-thiazol-2-ylamino]-4-methyl-phenyl}-4-(4-methyl-piperazin-1-ylmethyl)-benzamide

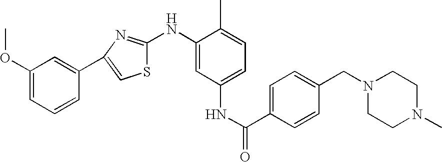

### 063: N-[4-Methyl-3-(4pyridin-3-yl-thiazol-2-ylamino)-phenyl]-isonicotinamide

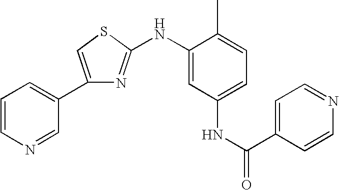

### 064: 2,6-Dichloro-N-[4methyl-3-(4-pyridin-3-yl-thiazol-2-ylamino)-phenyl]-isonicotinamide

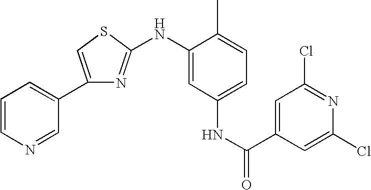

### 091: 3-Phenyl-propynoic acid [4-methyl-3-(4-pyridin-3-yl-thiazol-2-ylamino)-phenyl]-amide

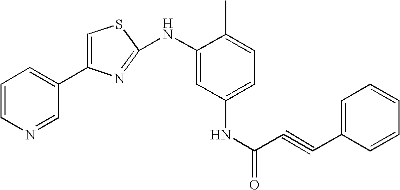

### 092: Cyclohexanecarboxylic acid [4-methyl-3-(4-pyridin-3-yl-thiazol-2-ylmethyl)-phenyl]-amide

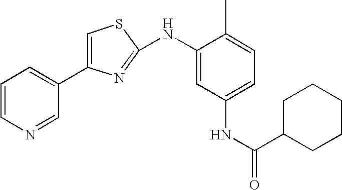

### 093: 5-[4Methyl-3-(4-pyridin-3-y-thiazol-2-ylamino)-phenylcarbamoyl]-pentanoic acid ethyl ester

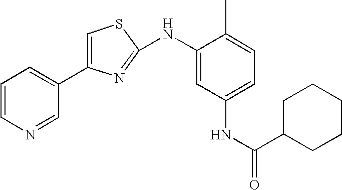

### 094: 1-Methyl-cyclohexanecarboxylic acid [4-methyl-3-(4-pyridin-3-yl-thiazol-2-ylmethyl)-phenyl]-amide

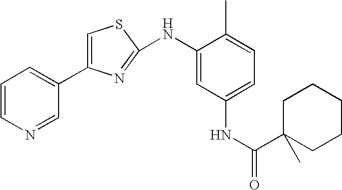

### 095: 4-tert-Butyl-cyclohexanecarboxylic acid [4-methyl-3-(4-pyridin-3-yl-thiazol-2-ylamino)-phenyl]-amide

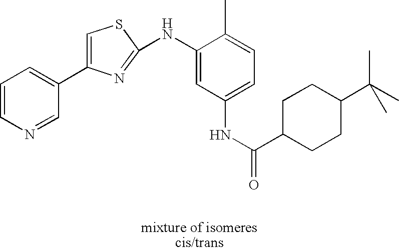

### 096: N-[4-Methyl-3-(4-pyridin-3-yl-thiazol-2-ylamino)-phenyl]-4-morpholin-4-yl-butyramide

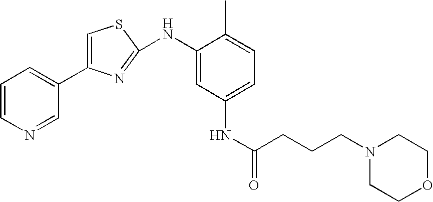

beige powder mp: 116-120° C.

¹H RMN (DMSO-d⁶) δ=1.80-2.00 (m, 2H); 2.29 (s, 3H); 2.30-2.45 (m, 6H); 3.55-3.65 (m, 6H); 7.15-7.25 (m, 2H); 7.46-7.50 (m, 2H); 7.52 (s, 1H); 8.35 (d, J=6.2 Hz, 1H); 8.55 (dd, J=1.5 Hz, J=4.7 Hz, 2H); 9.22 (s, 1H); 9.45 (s, 1H); 9.93 (s, 1H)

Among the compounds of formula IV, the invention is particularly embodied by the compounds wherein X is a urea group, a —CO—NRR′ group, corresponding to the [3-(thiazol-2-ylamino)-phenyl]-urea family and the following formula IV-2: 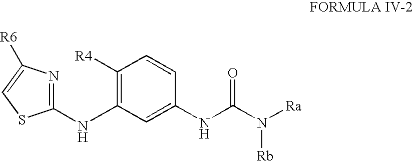
wherein Ra, Rb are independently chosen from H or an organic group that can be selected for example from a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with a heteroatom, notably a halogen selected from I, Cl, Br and F or bearing a pendant basic nitrogen functionality;
a —SO2-R group wherein R is an alkyl, cycloalkyl, aryl or heteroaryl optionally substituted with an heteroatom, notably a halogen selected from I, Cl, Br and F or bearing a pendant basic nitrogen functionality; or a —CO—R or a —CO—NRR′ group, wherein R and R′ are independently chosen from H, an alkyl, a cycloalkyl, an aryl or heteroaryl group optionally substituted with at least one heteroatom, notably selected from I, Cl, Br and F, or bearing a pendant basic nitrogen functionality.
R⁴ is hydrogen, halogen or a linear or branched alkyl group containing from 1 to 10 carbon atoms, trifluoromethyl or alkoxy;
R⁶ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.
iv) H, a halogen selected from I, F, Cl or Br, NH2, NO2 or SO2-R, wherein R is a linear or branched alkyl goup containing one or more group such as 1 to 10 carbon atoms, and optionally substituted with at least one heteroatom, notably a halogen selected from I, Cl, Br and F, and/or bearing a pendant basic nitrogen functionality.

### EXAMPLES

### 009: 1-(4-Methoxy-phenyl)-3-[4-methyl-3-(4-pyridin-3-yl-thiazol-2-ylamino)-phenyl]-urea

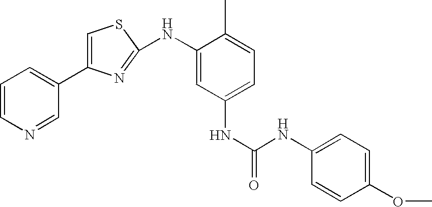

### 010: 1-(4-Bromo-phenyl)-3-[4-methyl-3-(4-pyridin-3-yl-thiazol-2-ylamino)-phenyl]-urea

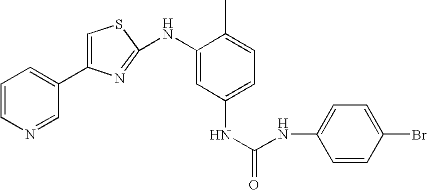

### 011: 1-[4-Methyl-3-(4-pyridin-3-yl-thiazol-2-ylamino)-phenyl]-3-(4-trifluoromethyl-phenyl)-urea

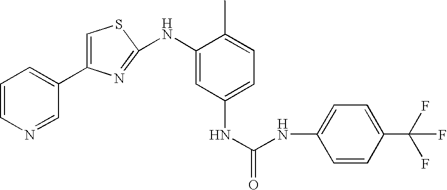

### 012: 1-(4-Fluoro-phenyl)-3-[4-methyl-3-(4-pyridin-3-yl-thiazol-2-ylamino)-phenyl]-urea

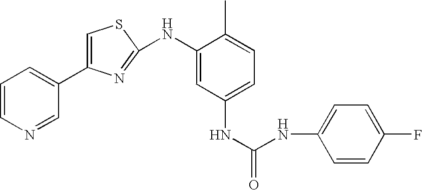

### 013: 1-[4-Methyl-3-(4-pyridin-3-yl-thiazol-2-ylamino)-phenyl]-3-(3,4,5-trimethoxy-phenyl)-urea

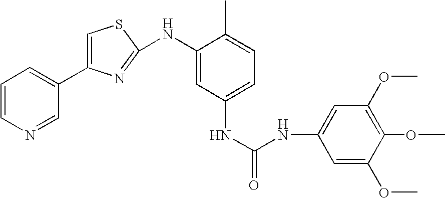

### 014: 4-{3-[4Methyl-3-(4-pyridin-3-yl-thiazol-2-ylamino)-phenyl]-ureido}-benzoic acid ethyl ester

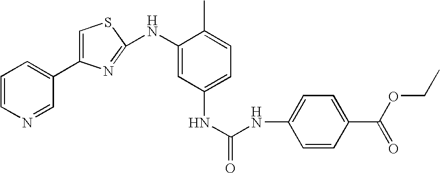

### 015: 1-[4-Methyl-3-(4-pyridin-3-yl-thiazol-2-ylamino)-phenyl]-3-thiophen-2-yl-urea

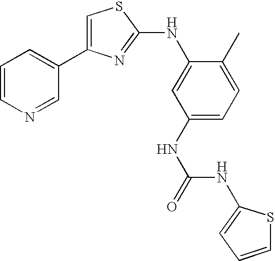

### 016: 1-Cyclohexyl-1-(N-Cyclohexyl-formamide)-3-[4-methyl-3-(4-pyridin-3-yl-thiazol-2-ylamino)-phenyl]-urea

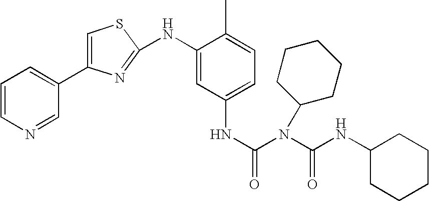

### 017: 1-(2,4-Dimethoxy-phenyl)-3-[4-methyl-3-(4-pyridin-3-yl-thiazol-2-ylamino)-phenyl]-urea

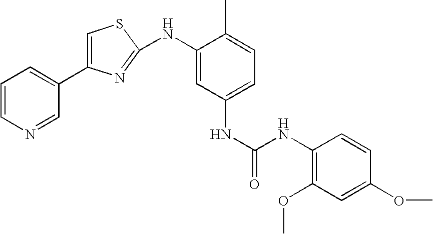

### 018: 1-(2-Iodo-phenyl)-1-(N-(2-Iodo-phenyl)-formamide)-3-[4-methyl-3-(4-pyridin-3-yl-thiazol-2-ylamino)-phenyl]-urea

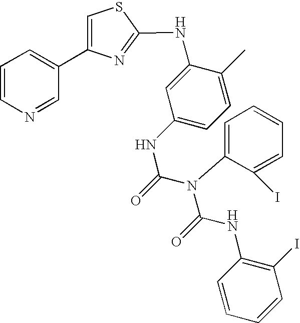

### 019: 1-(3,5-Dimethyl-isoxazol-4-yl)-3-[4methyl-3-(4-pyridin-3-yl-thiazol-2-ylamino)-phenyl]-urea

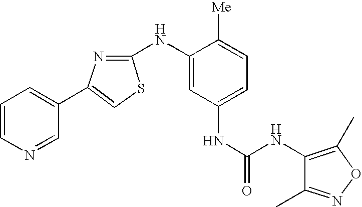

### 020: 1-(2-Iodo-phenyl)-3-[4-methyl-3-(4-pyridin-3-yl-thiazol-2-ylamino)-phenyl]-urea

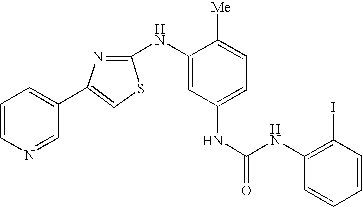

### 021: 1-(4-Difluoromethoxy-phenyl)-3-[4-methyl-3-(4-pyridin-3-yl-thiazol-2-ylamino)-phenyl]-urea

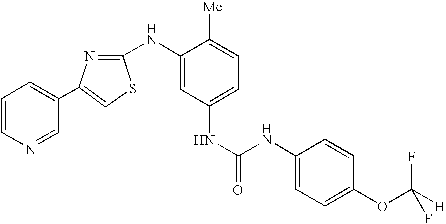

### 022: 1-(4-Dimethylamino-phenyl)-3-[4-methyl-3-(4-pyridin-3-yl-thiazol-2-ylamino)-phenyl]-urea

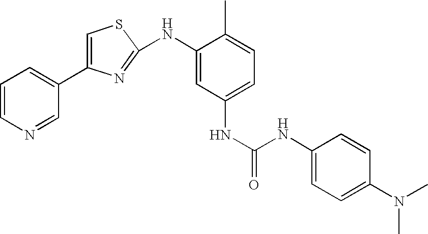

### 023: 1-(4-Fluoro-phenyl)-3-[4-methyl-3-(4-pyridin-3-yl-thiazol-2-ylamino)-phenyl]-urea

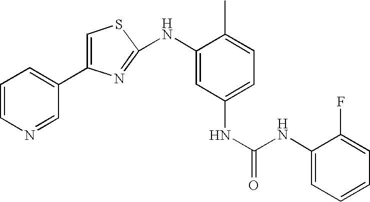

light brown powder mp: 203-206° C.

¹H NMR (DMSO-d⁶) : δ=2.24 (s, 3H); 6.98-7.00 (m, 2H); 7.10-7.23 (m, 314); 7.40 (m, 1H); 7.48 (s, 1H); 8.25 (m, 1H); 8.37 (d, J=7.8 Hz, 1H); 8.51 (m, 3H); 9.03 (s, 1H); 9.19 (s, 1H); 9.39 (s, 1H)

### 024: 1-(2-Chloro-phenyl)-3-[4-methyl-3-(4-pyridin-3-yl-thiazol-2-ylamino)-phenyl]-urea

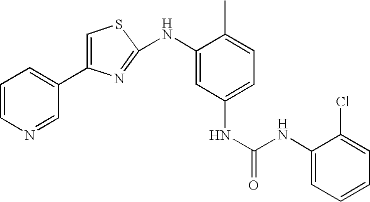

### 025: 1-(3-Fluoro-phenyl)-3-[4-methyl-3-(4-pyridin-3-yl-thiazol-2-ylamino)-phenyl]-urea

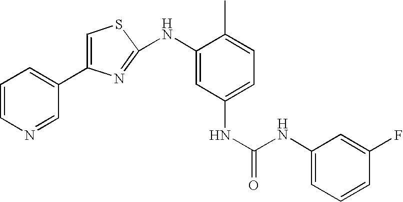

white powder mp: 210-215° C.

¹H NMR (DMSO-d⁶): δ 2.24 (s, 3H); 6.79 (t, J=6.3 Hz, 1H); 6.99 (m, 1H); 7.09-7.14 (m, 2H); 7.30 (m, 1H); 7.41 (t, J=4.7 Hz, 1H); 7.48 (s, 1H) ; 7.56 (d, J=1.2 Hz, 1H); 8.39 (d, J=8.0 Hz, 1H); 8.49-8.52 (m, 2H); 8.71 (s, 1H); 8.87 (s, 1H); 9.18 (s, 1H); 9.38 (s, 1H)

### 026: 1-[4Methyl-3-(4-pyridin-3-yl-thiazol-2-ylamino)-phenyl]-3-p-tolyl-urea

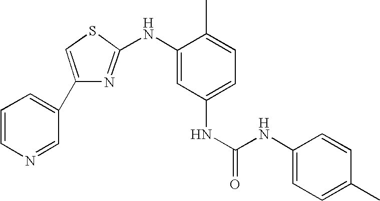

white powder mp: 210-215° C.

¹H RMN (DMSO-d⁶) δ=2.29 (s, 3H); 2.31 (s, 3H); 7.05 (d, J=6.2 Hz, 1H); 7.10-1.16 (m, 3H); 7.42-7.49 (m, 3H); 7.53 (s, 1H); 8.35-8.62 (m, 5H); 9.22 (d, J=1.6 Hz, 1H); 9.43 (s, 1H)

Among the compounds of formula IV, the invention is particularly embodied by the compounds wherein X is a -substituted Aryl group, corresponding to the N-[3-(Thiazol-2-ylamino)-phenyl]-amide family and the following formula IV-3: 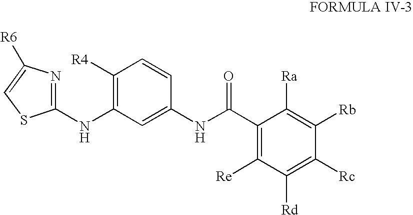
wherein Ra, Rb, Rc, Rd, Re are independently chosen from H or an organic group that can be selected for example from a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;
a —SO2-R group wherein R is an alkyl, cycloalkyl, aryl or heteroaryl optionally substituted with a heteroatom, notably a halogen selected from I, Cl, Br and F or bearing a pendant basic nitrogen functionality; or a —CO—R or a —CO—NRR′ group, wherein R and R′ are independently chosen from H, an alkyl, a cycloalkyl, an aryl or heteroaryl group optionally substituted with at least one heteroatom, notably selected from I, Cl, Br and F, and or bearing a pendant basic nitrogen functionality;
Ra, Rb, Rc, Rd, Re may also be
   a halogen such as I, Cl, Br and F
   a NRR′ group where R and R′ are H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;
   an OR group where R is H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality; a —SO2-R′ group wherein R′ is an alkyl, cycloalkyl, aryl or heteroaryl optionally substituted with a heteroatom, notably a halogen selected from I, Cl, Br and F or bearing a pendant basic nitrogen functionality;
   a NRaCORb group where Ra and Rb are H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;
   a NRaCONRbRc group where Ra and Rb are H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;
   a COOR, where R is a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality, or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
   a CONRaRb, where Ra and Rb are a hydrogen or a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
   an NHCOOR, where R is a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
   an OSO₂R, where R is a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality, or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
   an NRaOSO₂Rb, where Ra and Rb are a linear or branched alkyl group containing from 1 to 10 carbon atoms atoms optionally substituted with at least one heteroatom (for example a halogen) and/or bearing a pendant basic nitrogen functionality, Ra can also be a hydrogen; a cycloalkyl, an aryl or heteroaryl group optionally substituted with at least one heteroatom, notably a halogen selected from I Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from l, Cl, Br and F, and/or bearing a pendant basic nitrogen functionality;
   a CN group
   a trifluoromethyl group
R⁴ is hydrogen, halogen or a linear or branched alkyl group containing from 1 to 10 carbon atoms, trifluoromethyl or alkoxy;
R⁶ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy,
iv) H, a halogen selected from I, F, Cl or Br, NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality.

### EXAMPLES

### 028: 3-Bromo-N-[4-methyl-3-(4-pyridin-3-yl-thiazol-2-ylamino)-phenyl]-benzamide

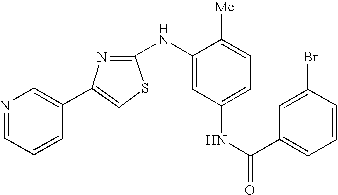

### 029: 3-Iodo-N-[4-methyl-3-(4-pyridin-3-yl-thiazol-2-ylamino)-phenyl]-benzamide

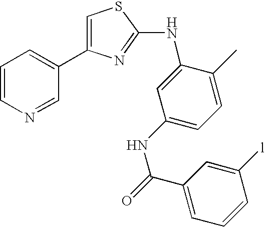

### 030: 4-Hydroxymethyl-N-[4-methyl-3-(4-pyridin-3-yl-thiazol-2-ylamino)-phenyl]-benzamide

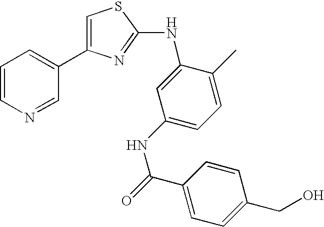

### 031: 4-Amino-N-[4-methyl-3-(4-pyridin-3-yl-thiazol-2-ylamino)-phenyl]-benzamide

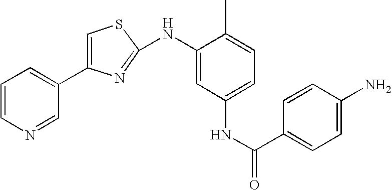

### 032: 2-Iodo-N-[4-methyl-3-(4-pyridin-3-yl-thiazol-2-ylamino)-phenyl]-benzamide

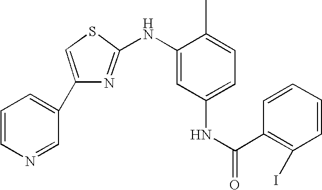

### 033: 4-Iodo-N-[4-methyl-3-(4-pyridin-3-yl-thiazol-2-ylamino)-phenyl]-benzamide

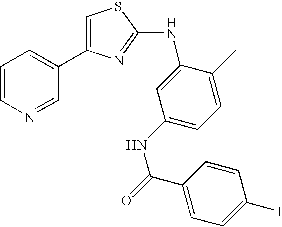

### 034: 4-(3-{4-[4-Methyl-3-(4-pyridin-3-yl-thiazol-2-ylamino)-phenylcarbamoyl]-phenyl}-ureido)-benzoic acid ethyl ester

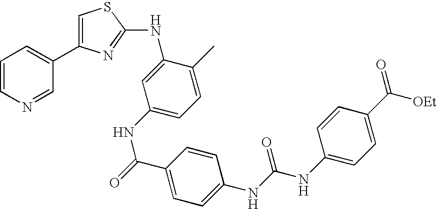

### 035: N-[4-Methyl-3-(4-pyridin-3-yl-thiazol-2-ylamino)-phenyl]-4-[3-(4-trifluoromethyl-phenyl)-ureido]-benzamide

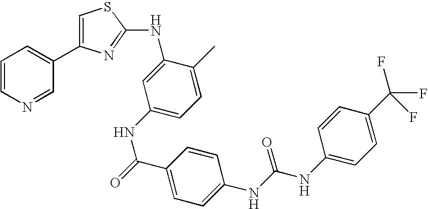

### 036: 4-[3-(4-Bromo-phenyl)-ureido]-N-[4-methyl-3-(4-pyridin-3-yl-thiazol-2-ylamino)-phenyl]-benzamide

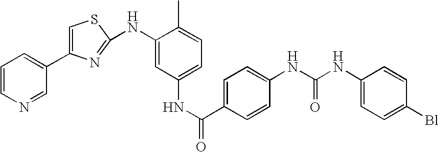

### 037: 4-Hydroxy-N-[4-methyl-3-(4-pyridin-3-yl-thiazol-2-ylamino)-phenyl]-benzamide

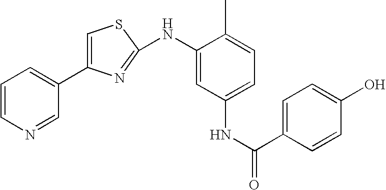

### 038: N-[4-Methyl-3-(4-pyridin-3-yl-thiazol-2-ylamino)-phenyl]-4-3-thiophen-2-yl-ureido)-benzamide

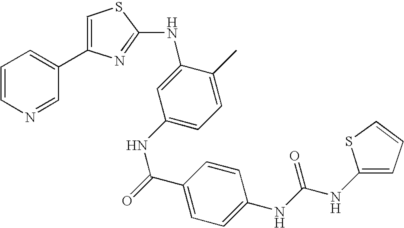

### 039: 4-[3-(3,5-Dimethyl-isoxazol-4-yl)-ureido]-N-[4-methyl-3-(4-pyridin-3-yl-thiazol-2-ylamino)-phenyl]-benzamide

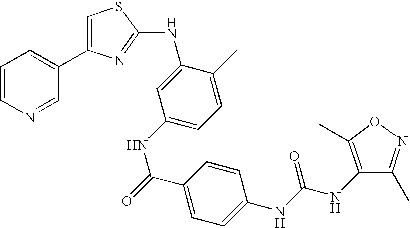

### 040: 4-[3-(4-Methoxy-phenyl)-ureido]-N-[4-methyl-3-(4-pyridin-3-yl-thiazol-2-ylamino)-phenyl]-benzamide

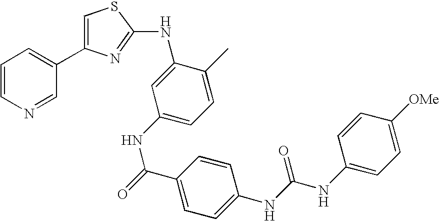

### 041: 4-[3-(4-Difluoromethoxy-phenyl)-ureido]-N-[4-methyl-3-(4-pyridin-3-yl-thiazol-2-ylamino)-phenyl]-benzamide

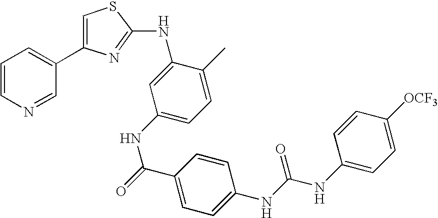

### 042: Thiophene-2-sulfonic acid 4-[4-methyl-3-(4-pyridin-3-yl-thiazol-2-ylamino)-phenylcarbamoyl]-phenyl ester

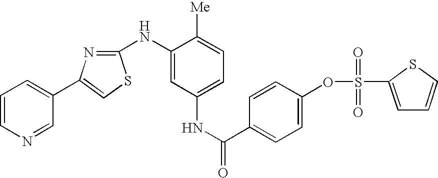

### 043: 4-Iodo-benzenesulfonic acid 4-[4-methyl-3-(4-pyridin-3-yl-thiazol-2-ylamino)-phenylcarbamoyl]-phenyl ester

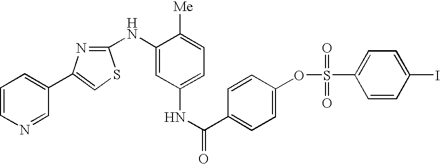

### 044: N-[4Methyl-3-(4-pyridin-3-yl-thiazol-2-ylamino)-phenyl]-4-(thiophene-2-sulfonylamino)-benzamide

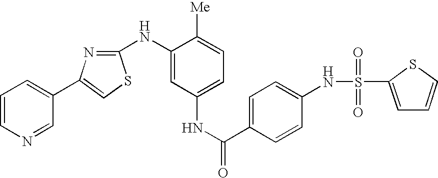

brown powder mp: 230-233° C.

¹H NMR (DMSO-d⁶) δ=2.29 (s, 3H); 7.15-7.18 (m, 2H); 7.22-7.32 (m, 3H); 7.48 (m, 2H); 7.67 (dd, J=1.3 Hz, J=3.7 Hz, 1H); 7.90-7.96 (m, 3H); 8.38-8.42 (m, 1H); 8.51 (m, 1H); 8.57 (d, J=1.9 Hz, 1H); 9.17 (d, J=1.7 Hz, 1H); 9.44 (s, 1H); 10.12 (s, 1H); 10.82 (s, 1H)

### 045: 3-Fluoro-N-[4-methyl-3-(4-pyridin-3-yl-thiazol-2-ylamino)-phenyl]-benzamide

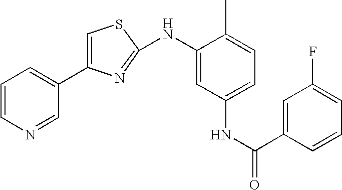

off-white foam mp: 184-186° C.

¹H NMR (CD₃OD-d⁴): =2.23 (s, 3H); 7.12-7.14 (m, 2H); 7.20-7.23 (m, 2H); 7.30 (m, 1H); 7.43 (m, 1H); 7.50 (m, 1H); 7.66 (d, J=1.0 Hz, 1H); 8.23 (m, 1H); 8.33 (m, 1H); 8.38 (s, 1H); 8.98 (s, 1H)

### 046: N-[4-Methyl-3-(4-pyridin-3-yl-thiazol-2-ylamino)-phenyl]-4-pyridin-4-yl-benzamide

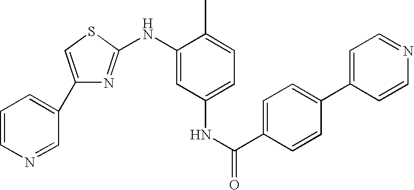

yellow powder mp: 254-256° C.

¹H NMR (DMSO-d⁶): δ2.34 (s, 3H); 7.28 (d, J=8.0 Hz, 1H); 7.45-7.49 (m, 2H); 7.54 (s, 1H); 7.78 (t, J=7.6 Hz, 1H); 7.89-7.91 (m, 2H); 8.10 (t, J=7.8 Hz, 2H); 8.37-8.42 (m, 2H); 8.55 (d, J=4.7 Hz, 1H); 8.73-8.77 (m, 3H); 9.24 (s, 1H); 9.52 (s, 1H); 10.43 (s, 1H)

### 047: 4-Dimethylamino-N-[4-methyl-3-(4-pyridin-3-yl-thiazol-2-ylamino)-phenyl]-benzamide

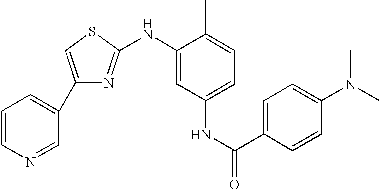

beige powder map: 147-150° C.

¹H NMR (DMSO-d⁶): 8 2.25 (s, 3H); 2.99 (s, 6H); 6.76 (d, J=8.9 Hz, 2H); 7.16 (d, J=8.3 Hz, 1H); 7.35 (d, J=2.0 Hz, 1H); 7.44-7.47 (m, 2H); 7.86-7.89 (m, 2H); 8.34-8.36 (m, 1H); 8.48-8.50 (m, 1H); 8.56-8.57 (m, 1H); 9.16 (s, 1H); 9.44 (s, 1H); 9.85 (s, 1H)

### 048: 2-Fluoro-5-methyl-N-[4-methyl-3-(4-pyridin-3-yl-thiazol-2-ylamino)-phenyl]-benzamide

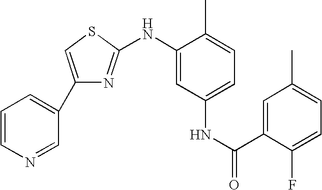

brown orange powder mp: 103-106° C.

¹H RMN (DMSO-⁶) δ=2.26 (s, 3H); 2.35 (s, 3H); 7.17-7.47 (m, 7H); 8.29 (dd, J=1.6 Hz, J=7.9 Hz, 1H); 8.47 (d, J=3.5 Hz, 1H); 8.57 (s, 1H); 9.15 (d, J=2.0 Hz, 1H); 9.44 (s, 1H); 10.33 (s, 1H)

### 049: 4-tert-Butyl-N-[4-methyl-3-(4-pyridin-3-yl-thiazol-2-ylamino)-phenyl]-benzamide

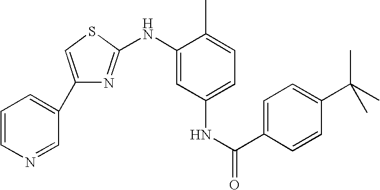

brown powder mp: 145-150° C.

¹H RMN (DMSO-d⁶) δ=1.32 (s, 9H); 2.04 (s, 3H); 7.18 (d, J=8.4 Hz, 1H); 7.35-7.44 (m, 2H); 7.46 (s, 1H); 7.55 (d, J=8.5 Hz, 1H); 7.90 (d, J=8.5 Hz, 1H); 8.32 (d, J=7.9 Hz, 1H); 8.47 (dd, J=1.5 Hz, J=4.7 Hz, 1H); 8.60 (d, J=2.0 Hz, 1H); 9.15 (d, J=1.7 Hz, 1H); 9.43 (s, 1H); 10.15 (s, 1H)

### 050: 4-Isopropoxy-N-[4-methyl-3-(4-pyridin-3-yl-thiazol-2-ylmethyl)-phenyl]-benzamide

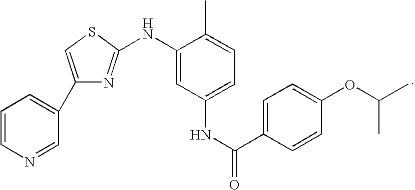

brown powder mp: 154-155° C.

¹H RMN (DMSO-d⁶) δ=1.34 (d, J=5.9 Hz, 6H); 4.72 (hept, J=5.9 Hz, 1H); 7.01 (d, J=7.0 Hz, 2H); 7.18 (d, J=8.5 Hz, 1H); 7.35-7.44 (m, 2H); 7.46 (s, 1H); 7.94 (dd, J=2.0 Hz, J=6.7 Hz, 2H); 8.32 (d, J=8.3 Hz, 1H); 8.48 (dd, J=3.3 Hz, J=4.8 Hz, 1H);8.58(d,J=2.0Hz, 1H);9.15 (d,J=1.8 Hz, 1H);9.43(s, 1H); 10.4 (s, 1H)

### 051: Benzo[1,3]dioxole-5-carboxylic acid [4-methyl-3-4-pyridin-3-yl-thiazol-2-ylmethyl)-phenyl]-amide

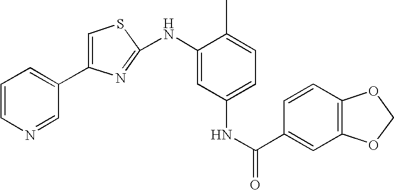

brown orange powder mp: 130-132° C.

¹H RMN (DMSO-d⁶) δ=2.23 (s, 3H); 6.10 (s, 2H); 7.03 (d, J=8.1Hz, 1H); 7.15 (d, J=8.3 Hz, 1H); 7.25-7.55 (m, 6H); 8.26 (s, 1H); 8.45 (dd, J=1.5 Hz, J=4.7, 1H); 8.55 (d, J=2.0 Hz, 1H); 9.12 (d, J=1.7 Hz, 1H); 9.40 (s, 1H); 10.01 (s, 1H)

### 052: N-[4-Methyl-3-(4-pyridin-3-yl-thiazol-2-ylamino)phenyl]-3-2-morpholin4-yl-ethoxy)-benzamide

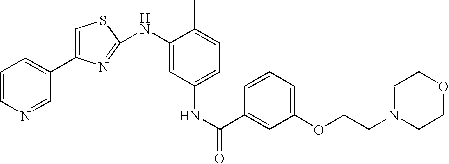

beige yellow powder mp: 75-80° C.

¹H RMN (DMSO-d⁶) δ=2.10-2.25 (m, 4H); 2.50-2.60 (m, 2H); 3.19 (s, 3H); 3.41-3.48 (m, 4H); 4.00-4.06 (m, 2H); 7.00-7.11 (m, 2); 7.22-7.35 (m, 6H), 8.18 (d, J=8.0 Hz, 1H); 8.33 (d, J=0.9 Hz, 1H); 8.49 (d, J=1.7 Hz, 1H); 9.03 (s, 1H); 9.31 (s, 1H); 10.05 (s, 1H)

### 053: N-[4-Methyl-3-(4-pyridin-3-yl-thiazol-2-ylmethyl)-phenyl]4-pyridin4-yl-benzamide

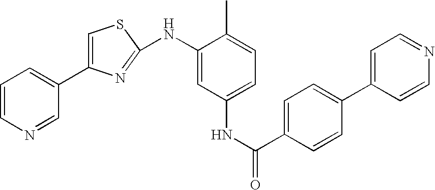

brown powder mp: dec. 250° C.

¹H RMN (DMSO-d⁶) δ=2.28 (s, 3H); 7.21 (d, J=7.9 Hz, 1H); 7.30-7.50 (m, 3H); 7.81 (d, J=4.7Hz, 1H); 7.98 (d, J=7.5 Hz, 2H); 8.13 (d, J=7.9 Hz, 2H); 8.32 (d, J=7.7 Hz, H); 8.48 (d, J=4.9 Hz, 1H); 8.62-8.69 (m, 3H); 9.16 (s, 1H); 9.45 (s, 1H); 10.34 (s, 1H)

### 054: 3-Cyano-N-[4-methyl-3-(4-pyridin-3-yl-thiazol-2-ylamino)-phenyl]-benzamide

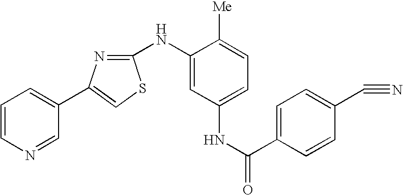

### 055: 2-Fluoro-N-[4methyl-3-(4-pyridin-3-yl-thiazol-2-ylamino)-phenyl]-3-trifluoromethyl-benzamide

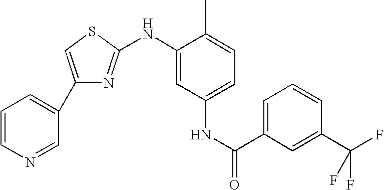

### 056: 3-Fluoro-benzenesulfonic acid 4-[4-methyl-3-(4-pyridin-3-yl-thiazol-2-ylamino)-phenylcarbamoyl]-phenyl ester

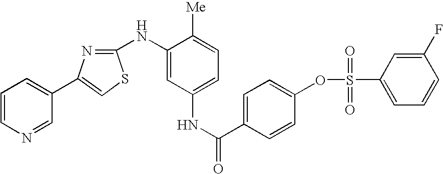

### 057: 4-Aminomethyl-N-[4-methyl-3-(4-pyridin-3-yl-thiazol-2-ylamino)-phenyl]-benzamide

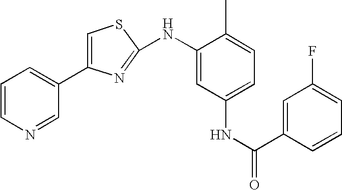

### 058: 2-Fluoro-benzenesulfonic acid 4-[4-methyl-3-(4-pyridin-3-yl-thiazol-2-ylamino)-phenylcarbamoyl]-phenyl ester

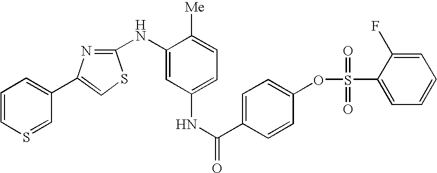

### 059: 3-Methoxy-N-[4-methyl-3-(4-pyridin-3-yl-thiazol-2-ylmethyl)-phenyl]-benzamide

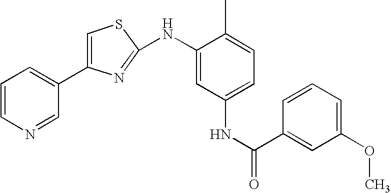

white powder mp: 76-79° C.

¹H RMN (DMSO-⁶) δ=2.32 (s, 3H); 3.89 (s, 3H); 7.22-7.25 (m, 2H), 7.44-7.58 (m, 4H), 8.28-8.35 (m, 1H); 8.52 (dd, J=1.6 Hz, J=4.7 Hz, 1H); 8.66 (d, J=2.0 Hz, 1H); 9.20 (d, J=1.4 Hz, 1H); 9.50 (s, 1H); 10.25 (s, 1H)

### 060: 4-(4-Methyl-piperazin-1-yl)-N-[4-methyl-3-(4-pyridin-3-yl-thiazol-2-ylmethyl)-phenyl]-benzamide

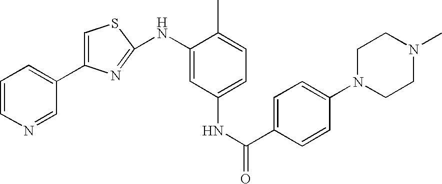

beige brown powder mp: 128-130° C.

¹H RMN (DMSO-d⁶) δ=2.15 (s, 3H); 2.18 (s, 3H); 2.35-2.41 (m, 4H); 3.18-3.24 (m, 4H); 6.94 (d, J=8.9 Hz, 2H); 7.09 (d, J=8.4 Hz, 1H); 7.28-7.38 (m, 3H); 7.81 (d, J=8.9 Hz, 2H); 8.20-8.25 (m, 1H); 8.40 (dd, J=1.6 Hz, J=4.7, 1H); 8.48 (d, J=1.9 Hz, 1H); 9.07 (d, J=1.5 Hz, 1H); 9.35 (s, 1H); 9.84 (s, 1H)

### 061: 3-Methyl-N-[4-methyl-3-(4-pyridin-3-yl-thiazol-2-ylamino)-phenyl]-benzamide

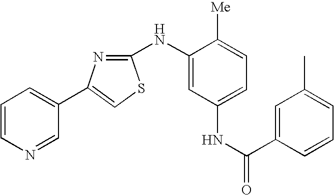

### 062: Biphenyl-3-carboxylic acid [4-methyl-3-(4-pyridin-3-yl-thiazol-2-ylamino)-phenyl]-amide

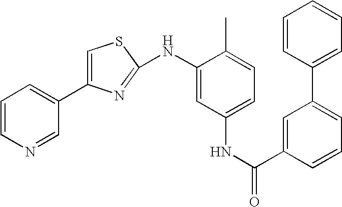

### 065: N-[4Methyl-3-(4-pyridin-3-yl-thiazol-2-ylamino)-phenyl]-3-trifluoromethyl-benzamide

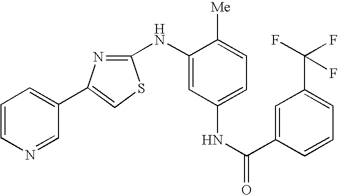

### 099: N-[4-Methyl-3-(4-pyridin-3-yl-thiazol-2-ylamino)-phenyl]4-pyrrolidin-1-ylmethyl-benzamide

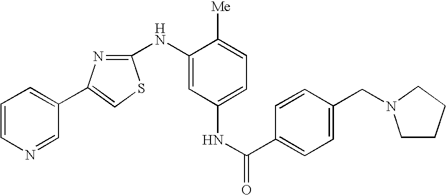

### 100: 4-[3-(2,4-Dimethoxy-phenyl)-ureido]-N-[4-methyl-3-(4-pyridin-3-yl-thiazol-2-ylamino)-phenyl]-benzamide

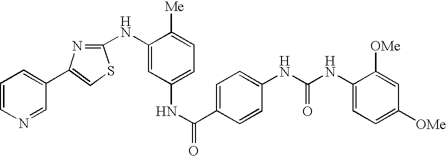

### 101: 4-[3-(2-Iodo-phenyl)-ureido]-N-[4-methyl-3-(4-pyridin-3-yl-thiazol-2-ylamino)-phenyl]-benzamide

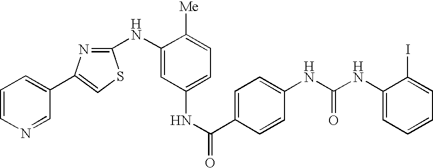

### 102: 4-[3-4-Fluoro-phenyl)-ureido]-N-[4-methyl-3-(4-pyridin-3-yl-thiazol-2-ylamino)-phenyl]-benzamide

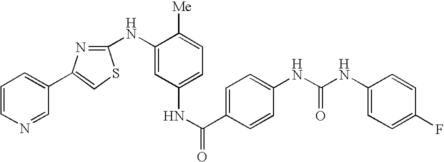

### 105: 3-Bromo4-methyl-N-[4-methyl-3-(4-pyridin-3-yl-thiazol-2-ylamino)-phenyl]-benzamide

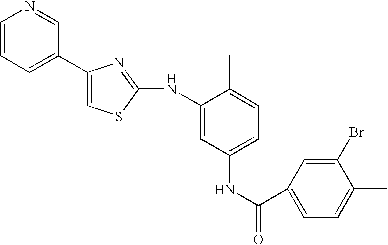

### 106: 4-Fluoro-N-[4-methyl-3-(4-pyridin-3-yl-thiazol-2-ylamino)-phenyl]-benzamide

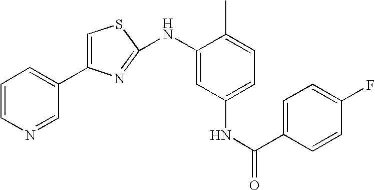

### 103: 4-Cyano-N-[4methyl-3-(4-pyridin-3-yl-thiazol-2-ylamino)-phenyl]-benzamide

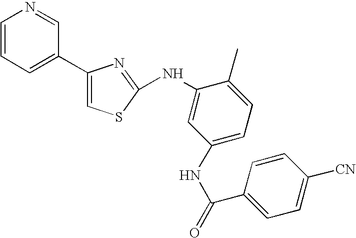

### 104: 4-Fluoro-N-[4-methyl-3-(4-pyridin-3-yl-thiazol-2-ylamino)-phenyl]-benzamide

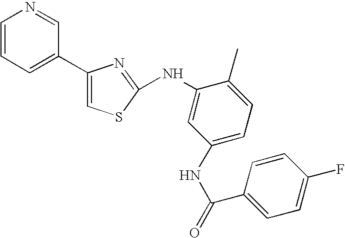

Among compounds of formula IV, the invention is particularly embodied by the compounds wherein X is a -substituted-aryl group, corresponding to the 4-(4-substituted-1-ylmethyl)-N-[3-thiazol-2-ylamino)-phenyl]-benzamide family and the following formula IV-4: 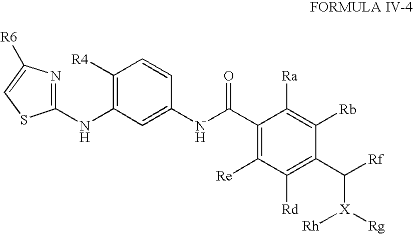
wherein X is a heteroatom, such as O or N
wherein Ra, Rb, Rd, Re, Rf, Rg, Rh are independently chosen from H or an organic group that can be selected for example from a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;
   or a NRR′ group where R and R′ are H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;
   or an OR group where R is H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality; a —SO2-R′ group wherein R′ is an alkyl, cycloalkyl, aryl or heteroaryl optionally substituted with a heteroatom, notably a halogen selected from I, Cl, Br and F or bearing a pendant basic nitrogen functionality;
   or a NRaCORb group where Ra and Rb are H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloallyl, an aryl or heteroaryl group optionally substituted with an heteroatom, notably a halogen selected from I, Cl, Br and F or bearing a pendant basic nitrogen functionality;
   or a NRaCONRbRc group where Ra and Rb are H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;
   or a COOR, where R is a linear or branched alkyl group containing from 1 to 10 carbon atoms atoms optionally substituted with at least one heteroatom (for example a halogen) and/or bearing a pendant basic nitrogen functionality, a cycloalkyl, an aryl or heteroaryl group optionally substituted with at least one heteroatom, notably a halogen selected from I, Cl, Br and F, and/or bearing a pendant basic nitrogen fuictionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
   or a CONRaRb, where Ra and Rb are a hydrogen or a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
   or an NHCOOR, where R is a linear or branched alkyl group containing from 1 to 10 carbon atoms atoms optionally substituted with at least one heteroatom (for example a halogen) and/or bearing a pendant basic nitrogen functionality; a cycloallyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
   an OSO₂R, where R is a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
   or an NRaOSO₂Rb, where Ra and Rb are a linear or branched alkyl group containing from 1 to 10 carbon atoms atoms optionally substituted with at least one heteroatom (for example a halogen) and/or bearing a pendant basic nitrogen functionality; Ra can also be a hydrogen; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
   or a —SO₂-R group wherein R is an alkyl, cycloalkyl, aryl or heteroaryl optionally substituted with an heteroatom, notably a halogen selected from I, Cl, Br and F or bearing a pendant basic nitrogen functionality; or a —CO—R or a —CO—NRR′ group, wherein R and R′ are independently chosen from H, an alkyl, a cycloalkyl, an aryl or heteroaryl group optionally substituted with at least one heteroatom, notably selected from I, Cl, Br and F, and/or bearing a pendant basic nitrogen functionality.
Ra, Rb, Rd, Re can also be halogen such as Cl, F, Br, I or trifluoromethyl;
R⁴ is hydrogen, halogen or a linear or branched alkyl group containing from 1 to 10 carbon atoms, trifluoromethyl or alkoxy;
R⁶ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy;
iv) H, a halogen selected from I, F, Cl or Br, NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality.

### EXAMPLES

### 066: 4-(4-methyl-piperazin-1-ylmethyl)-N-[4-methyl-3-(4-pyridin-3-yl-thiazol-2-ylamino)-phenyl]-benzamide

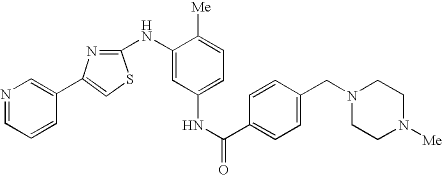

### 067: 3,5-Dibromo-4-(4-methyl-piperazin-1-ylmethyl)-N-[4-methyl-3-(4-pyridin-3-yl-thiazol-2-ylamino)-phenyl]-benzamide

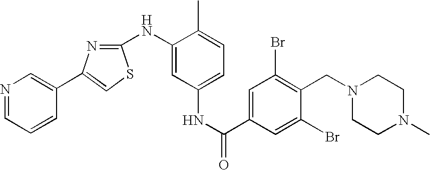

### 068: 4-Diethylaminomethyl-N-[4-methyl-3-(4-pyridin-3-yl-thiazol-2-ylamino)-phenyl]-benzamide

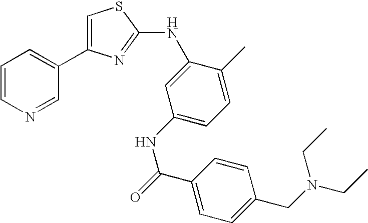

### 069: N-[4-Methyl-3-(4-pyridin-3-yl-thiazol-2-ylamino)-phenyl]-4-morpholin-4-ylmethyl-benzamide

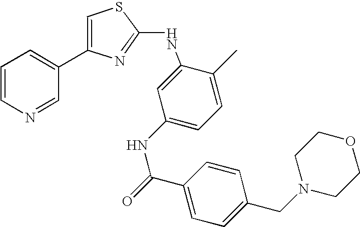

### 070: 4-Dipropylaminomethyl-N-[4-methyl-3-(4-pyridin-3-yl-thiazol-2-ylamino)-phenyl]-benzamide

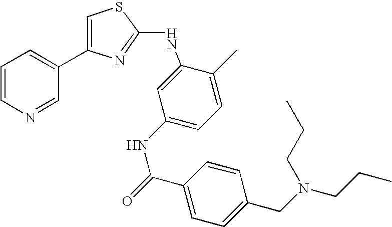

### 071: N-[4-Methyl-3-(4-pyridin-3-yl-thiazol-2-ylamino)-phenyl]-4-piperidin-1-ylmethyl-benzamide

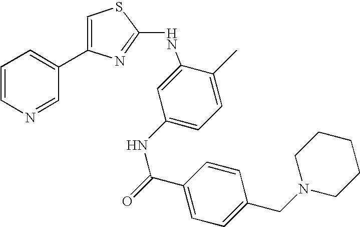

### 072: 4-[Diisopropylamino)-methyl]-N-[4-methyl-3-(4-pyridin-3-yl-thiazol-2-ylamino)-phenyl]-benzamide

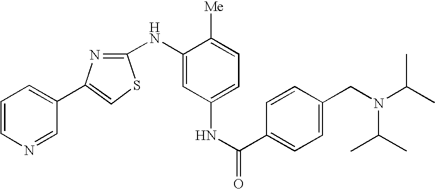

### 073: {4-[4-Methyl-3-(4-pyridin-3-yl-thiazol-2-ylamino)-phenylcarbamoyl]-benzyl}-carbamic acid tert-butyl ester

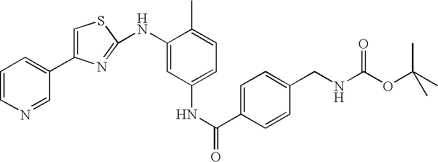

### 074: 3-Fluoro4-4-(4-methyl-piperazin-1-ylmethyl)-N-[4-methyl-3-(4-pyridin-3-yl-thiazol-2-ylamino)-phenyl]-benzamide

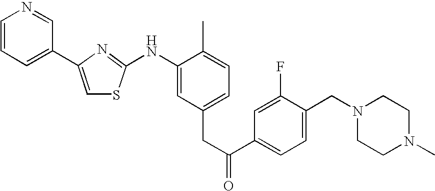

### 075: 4-(4-Methyl-piperazin-1-ylmethyl)-N-[4-methyl-3-(4-pyridin-3-yl-thiazol-2-ylmethyl)-phenyl]-3-trifluoromethyl-benzamide

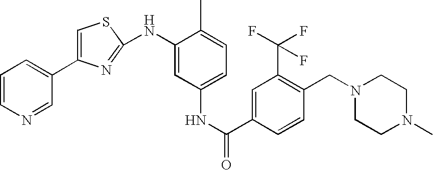

yellow Crystals mp: 118-120° C.

¹H RMN (DMSO-d⁶) δ=2.22 (s, 3H); 2.33 (s, 3H); 2.34-2.50 (m, 8H); 3.74 (s, 2H)) 7.26 (d, J=8.3Hz, 1H); 7.41-7.49 (m, 2H); 7.53 (s, 1H); 7.99 (d, J=8.0 Hz, 1H); 8.28-8.31 (m, 2H); 8.38 (d, J=7.9 Hz, 1H); 8.53 (dd, J=1.3 Hz, J=4.7 Hz, 1 H); 8.68 (d, J=1.9 Hz, 1H); 9.21 (d, J=2.0 Hz, 1H); 9.53 (s, 1H); 10.49 (s, 1H)

### 076: 2,3,5,6-Tetrafluoro-4-(4-methyl-piperazin-1-ylmethyl)-N-[4-methyl-3-(4-pyridin-3-yl-thiazol-2-ylamino)-phenyl]-benzamide

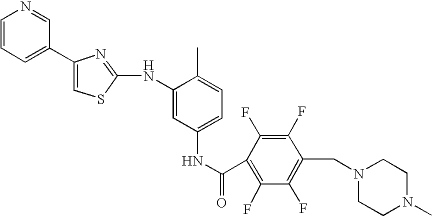

### 077: N-{3-[4-(4-Fluoro-phenyl)-thiazol-2-ylamino]-4-methyl-phenyl}-4-(4-methyl-piperazin-1-ylmethyl)-benzamide

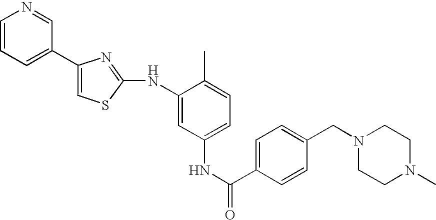

### 078: 3-Bromo-4-(4-methyl-piperazin-1-ylmethyl)-N-[4-methyl-3-(4-pyridin-3-yl-thiazol-2-ylamino)-phenyl]-benzamide

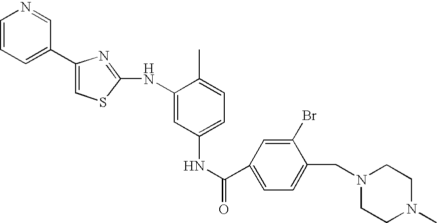

### 079: 3-Chloro-4-(4-methyl-piperazin-1-ylmethyl)-N-[4-methyl-3-(4-pyridin-3-yl-thiazol-2-ylamino)-phenyl]-benzamide

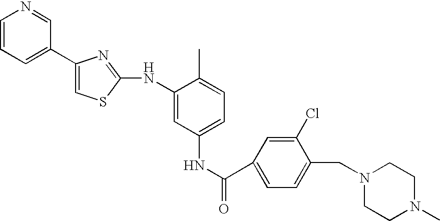

### 080: 4-(4-Methyl-piperazin-1-ylmethyl)-N-[4-methyl-3-(4-pyridin-4-yl-thiazol-2-ylamino)-phenyl]-benzamide

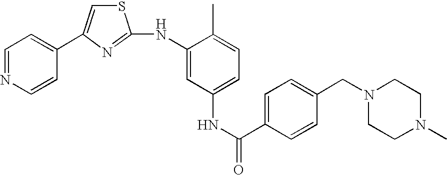

### 081: N-{3-[4-(4-Cyano-phenyl)-thiazol-2-ylamino]-4-methyl-phenyl}-4-(4-methyl-piperazin-1-ylmethyl)-benzamide

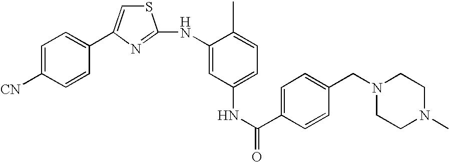

### 082: 4-[1-(4-Methyl-piperazin-1-yl)-ethyl]-N-[4-methyl-3-(4-pyridin-3-yl-thiazol-2-ylmethyl)-phenyl]-benzamide

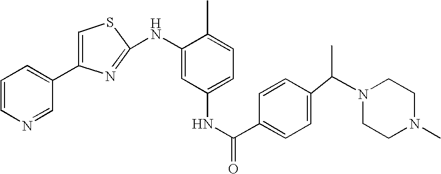

beige powder mp: 153-155° C.

¹H RMN (DMSO-d⁶) δ=1.29 (d, J=6.6 Hz, 3H); 2.15 (s, 3H); 2.26 (s, 3H); 3.15-3.25 (m, 9H); 7.18 (d, J=8.4Hz, 1H); 7.35-7.47 (m, 5H); 7.91 (d, J=8.2Hz, 2H); 8.31 (d, J=8.0 Hz, 1H); 8.47 (dd, J=1.6 Hz, J=4.7 Hz, 1H); 8.60 (d, J=2.0, 1H); 9.15 (d, J=0.6, 1H); 9.45 (s, 1H); 10.18 (s, 1H)

### 083: 4-(1-Methoxyethyl)-N-[4-methyl-3-(4-pyridin-3-yl-thiazol-2-ylmethyl)-phenyl]-benzamide

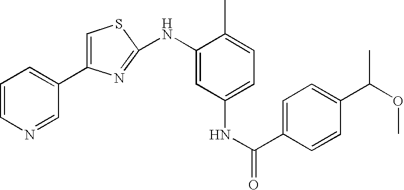

### 084: N-{4-Methyl-3-[4-(5-methyl-pyridin-3-yl)-thiazol-2-ylamino]-phenyl}-4-(4-methyl-piperazin-1-ylmethyl)-benzamide

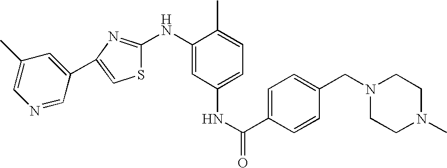

### 085: 3-Iodo-4-(4-methyl-piperazin-1-ylmethyl)-N-[4-methyl-3-(4-pyridin-3-yl-thiazol-2-ylmethyl)-phenyl]-benzamide

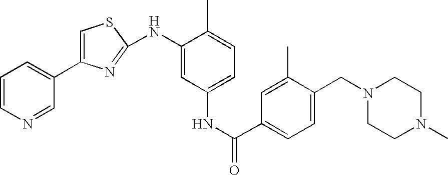

### 086: N-[4-Methyl-3-(4-pyridin-3-yl-thiazol-2-ylamino)-phenyl]-4-[3-(4-trifluoromethyl-phenyl)-ureidomethyl]-benzamide

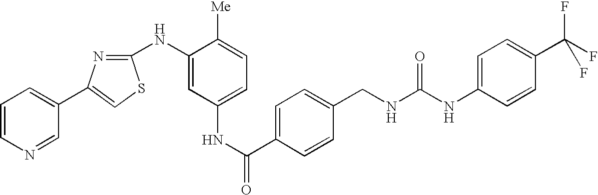

### 087: 3,5-Dibromo-N-[4-methyl-3-(4-pyridin-3-yl-thiazol-2-ylamino)-phenyl]-4-[(3-morpholin-4-yl-propylamino)-methyl]-benzamide

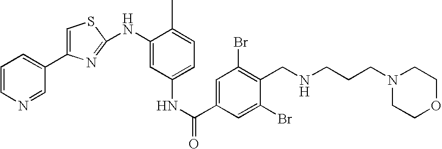

### 107: 3,5-Dibromo-N-[4-methyl-3-(4-pyridin-3-yl-thiazol-2-ylamino)-phenyl]-4-piperidin-1-ylmethyl-benzamide

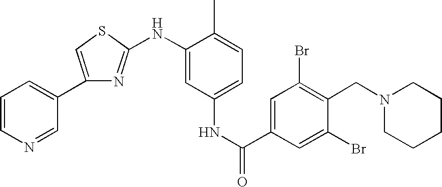

### 122: 4-(4-Methyl-piperazin-1-ylmethyl)-N-[4-methyl-3-(4-pyridin-2-yl-thiazol-2-ylamino)-phenyl]-benzamide

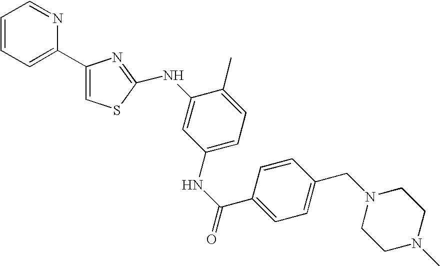

### 111: N-{3-[4-(3-Fluoro-phenyl)-thiazol-2-ylamino]-4-methyl-phenyl}-4-(4-methyl-piperazin-1-ylmethyl)-benzamide

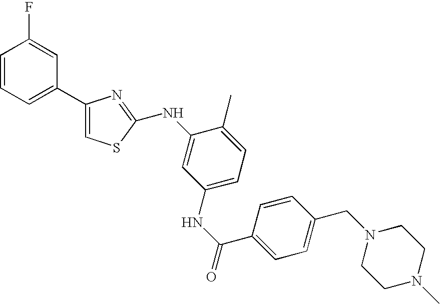

### 118: N-{3-[4-(2-Fluoro-phenyl)-thiazol-2-ylamino]-4-methyl-phenyl}-4-(4-methyl-piperazin-1-ylmethyl)-benzamides

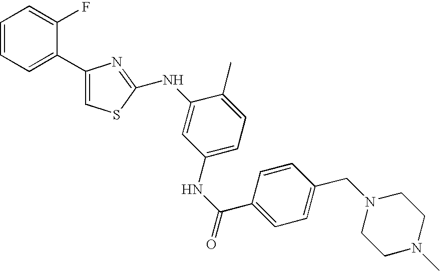

Among compounds of formula IV, the invention is particularly embodied by the compounds wherein X is a -aryl-substituted group, corresponding to the 3-Disubstituted-amino-N[-3-(thiazol-2-ylamino)-phenyl]-benzamide family and the following formula IV-5: 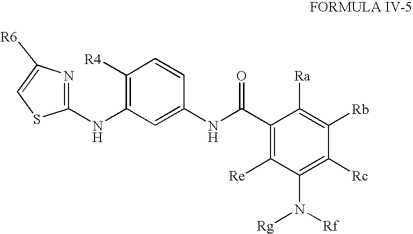
wherein Ra, Rb, Rc, Re, Rf, Rg are independently chosen from H or an organic group that can be selected for example from a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;
or a NRR′ group where R and R′ are H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;
or an OR group where R is H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality; a —SO2-R′ group wherein R′ is an alkyl, cycloalkyl, aryl or heteroaryl optionally substituted with a heteroatom, notably a halogen selected from I, Cl, Br and F or bearing a pendant basic nitrogen functionality;
or a NRaCORb group where Ra and Rb are H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;
or a NRaCONRbRc group where Ra and Rb are H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;
or a COOR, where R is a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
or a CONRaRb, where Ra and Rb are a hydrogen or a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
or an NHCOOR, where R is a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an OSO₂R, where R is a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
or an NRaOSO₂Rb, where Ra and Rb are a linear or branched alkyl group containing from 1 to 10 carbon atoms atoms optionally substituted with at least one heteroatom (for example a halogen) and/or bearing a pendant basic nitrogen functionality; Ra can also be a hydrogen; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
or a —SO2-R group wherein R is an alkyl, cycloalkyl, aryl or heteroaryl optionally substituted with an heteroatom, notably a halogen selected from I, Cl, Br and F or bearing a pendant basic nitrogen functionality; or a —CO—R or a —CO—NRR′ group, wherein R and R′ are independently chosen from H, an alkyl, a cycloalkyl, an aryl or heteroaryl group optionally substituted with at least one heteroatom, notably selected from I, Cl, Br and F, and/or bearing a pendant basic nitrogen functionality.
Ra, Rb, Rc, Re can also be halogen such as Cl, F, Br, I or trifluoromethyl;
R⁴ is hydrogen, halogen or a linear or branched alkyl group containing from 1 to 10 carbon atoms, trifluoromethyl or alkoxy;
R⁶ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy;
iv) H, a halogen selected from I, F, Cl or Br; NH2, NO2 or SO2-R, wherein R is a linear or branched alkyl goup containing one or more group such as 1 to 10 carbon atoms, and optionally substituted with at least one heteroatom, notably a halogen selected from I, Cl, Br and F, and/or bearing a pendant basic nitrogen functionality.

### EXAMPLES

### 088: 3-Dimethylamino-N-[4-methyl-3-(4-pyridin-3-yl-thiazol-2-ylamino)-phenyl]-benzamide

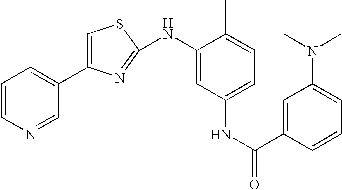

beige powder mp: 197-198° C.

¹H NMR (DMSO-d⁶): δ=2.32 (s, 3H); 3.03 (s, 6H); 6.97 (d, J=6.4 Hz, 1H); 7.23-7.56 (m, 7H); 8.37 (d, J=7.3 Hz, 1H); 8.53 (d, J=4.7 Hz, 1H); 8.63 (s, 1H); 9.20 (s, 1H); 9.48 (s, 1H); 10.15 (s, 1H)

### 089: 3-(4-Methyl-piperazin-1-yl)-N-[4-methyl-3-(4-pyridin-3-yl-thiazol-2-ylamino)-phenyl]-benzamide

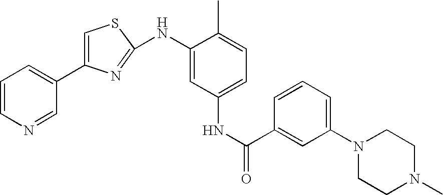

beige powder mp: 274-246° C.

₁H RMN (DMSO-d⁶) δ=2.23 (s, 3H); 2.24-2.30 (m, 4H); 3.22-3.27 (m, 4H); 7.07-7.20 (m, 2H); 7.36-7.53 (m, 6H); 8.31 (d, J=7.5 Hz, 1H); 8.47 (d, J=3.7 Hz, 1H); 8.58 (s, 1H); 9.12 (d, J=7.8 Hz, 1H); 9.44 (s, 1H); 10.12 (s, 1H)

### 090: N-[4-Methyl-3-(4-pyridin-3-yl-thiazol-2-ylamino)-phenyl]-3-morpholin-4-yl-benzamide

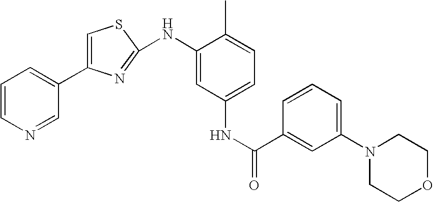

beige powder mp: 247-248° C.

¹H RMN (CDCl₃) δ=1.50 (s, 3H); 3.15-3.18 (m, 4H); 3.79-3.82 (m, 3H); 6.85 (s, 1H); 7.00-7.30 (m, 7H); 7.41 (s, 1H); 7.75 (s, 1H); 8.08 (d, J=7.9 Hz, 1H); 8.22 (d, J=1.7 Hz, 1H); 8.46 (dd, J=1.3 Hz, J=4.7 Hz, 1H); 9.01 (d, J=1.6 Hz, 1H)

Among the compounds of formula IV, the invention is particularly embodied by the compounds wherein X is a —OR group, corresponding to the family [3-(Thiazol-2-ylamino)-phenyl]-carbamate and the following formula IV-6 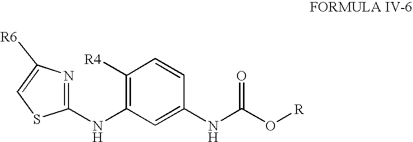
wherein R is independently chosen from an organic group that can be selected for example from a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with an heteroatom, notably a halogen selected from I, Cl, Br and F and/or bearing a pendant basic nitrogen functionality; or a cycloalkyl, an aryl or heteroaryl group optionally substituted with a cycloalkyl, an aryl or heteroaryl group optionally substituted with a heteroatom, notably a halogen selected from I, Cl, Br and F and/or bearing a pendant basic nitrogen functionality;
R⁴ is hydrogen, halogen or a linear or branched alkyl group containing from 1 to 10 carbon atoms, trifluoromethyl or alkoxy;
R⁶ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy; s
iv) H, a halogen selected from I, F, Cl or Br; NH2, NO2 or SO2-R, wherein R is a linear or branched alkyl goup containing one or more group such as 1 to 10 carbon atoms, and optionally substituted with at least one heteroatom, notably a halogen selected from I, Cl, Br and F, and/or bearing a pendant basic nitrogen functionality.

### EXAMPLES

### 097: [4-Methyl-3-(4-pyridin-3-yl-thiazol-2-ylamino)-phenyl]-carbamic acid isobutyl ester

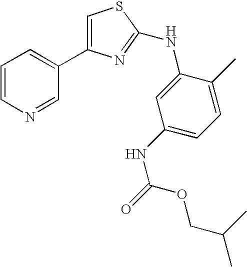

### 098: 2-(2-methyl-5-tert-butoxycarbonylamino)phenyl-4-(3-pyridyl)-thiazole

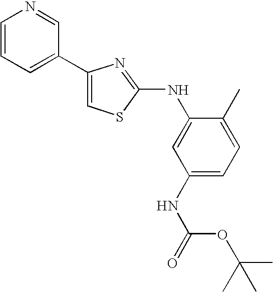 Process for Manufacturing a Compound of Formula m depicted above.

This entails the condensation of a substrate of general formula 10 with a thiourea of the type 11.

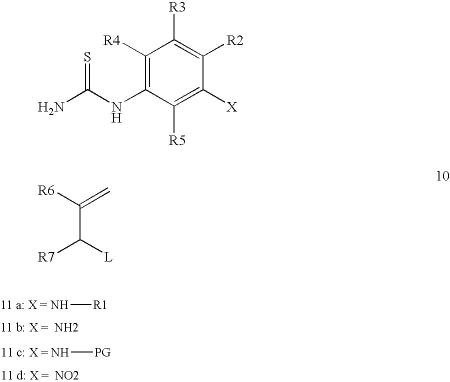

Substituent “L” in formula 10 is a nucleofugal leaving group in nucleophilic substitution reactions (for example, L can be selected from chloro, bromo, iodo, toluenesulfonyloxy, methanesulfonyloxy, trifluoromethanesulfonyloxy, etc., with L being preferentially a bromo group).

Group R1 in formula 11a corresponds to group R1 as described in formula III.

Group “PG” in formula 11c is a suitable protecting group of a type commonly utilized by the person skilled in the art.

The reaction of 10 with 1 a-d leads to a thiozole-type product of formula 12a-d.

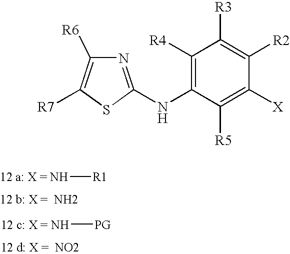

Formula 12a is the same as formula I. Therefore, R1 in 12a corresponds to R1 in formula III.

Formula 12b describes a precursor to compounds of formula III which lack substituent R1. Therefore, in a second phase of the synthesis, substituent R1 is connected to the free amine group in 12b, leading to the complete structure embodied by formula III:
12b+“R1”→III
The introduction of R1, the nature of which is as described on page 3 for the general formula III, is achieved by the use of standard reactions that are well known to the person skilled in the art, such as alkylation, acylation, sulfonylation, formation of ureas, etc.

Formula 12c describes an N-protected variant of compound 12b. Group “PG” in formula 12c represents a protecting group of the type commonly utilized by the person skilled in the art. Therefore, in a second phase of the synthesis, group PG is cleaved to transform compound 12c into compound 12b. Compound 12b is subsequently advanced to structures of formula I as detailed above.

Formula 12d describes a nitro analogue of compound 12b. In a second phase of the synthesis, the nitro group of compound 12d is reduced by any of the several methods utilized by the person skilled in the art to produce the corresponding amino group, namely compound 12b. Compound 12b thus obtained is subsequently advanced to structures of formula III as detailed above.

### Examples of Compound Synthesis

General: All chemicals used were commercial reagent grade products. Dimethylformamide (DMF), methanol (MeOH) were of anhydrous commercial grade and were used without further purification. Dichloromethane and tetrahydrofuran (THF) were freshly distilled under a stream of argon before use. The progress of the reactions was monitored by thin layer chromatography using precoated silica gel 60F 254, Fluka TLC plates, which were visualized under UV light. Multiplicities in ¹HNMR spectra are indicated as singlet (s), broad singlet (br s), doublet (d), triplet (t), quadruplet (q), and multiplet (m) and the NMR spectrum were realized on a 300 MHz Bruker spectrometer.

### 3-Bromoacetyl-pyridine, HBr salt

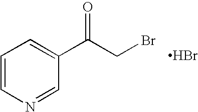

Dibromine (17.2 g, 108 mmol) was added dropwise to a cold (0° C.) solution of 3-acetyl-pyridine (12 g, 99 mmol) in acetic acid containing 33% of HBr (165 mL) under vigourous stirring. The vigorously stirred mixture was warmed to 40° C. for 2 h and then to 75° C. After 2h at 75° C., the mixture was cooled and diluted with ether (400 mL) to precipitate the product which was recovered by filtration and washed with ether and acetone to give white crystals (100%). This material may be recrystallised from methanol and ether.

IR (neat): 3108, 2047, 2982, 2559, 1709, 1603, 1221, 1035, 798 cm⁻¹-¹H NMR (DMSO-d⁶) δ=5.09 (s, 2H, CH₂Br); 7.88 (m, 1H, pyridyl-H); 8.63 (m, 1H, pyridyl-H); 8.96 (m, 1H, pyridyl-H); 9.29 (m, 1H, pyridyl-H).

### Methyl-[4(1-N-methyl-piperazino)-methyl]-benzoate

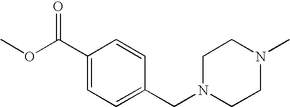

To methyl-4-formyl benzoate (4.92 g, 30 mmol) and N-methyl-piperazine (3.6 mL, 32 mmol) in acetonitrile (100 mL) was added dropwise 2.5 mL of trifluoroacetic acid. The reaction mixture was stirred at room temperature for 1 h. After slow addition of sodium cyanoborohydride (2 g, 32 mmol), the solution was left stirring overnight at room temperature. Water (10 mL) was then added to the mixture, which was further acidified with 1N HCl to pH=6-7. The acetonitrile was removed under reduced pressure and the residual aqueous solution was extracted with diethyl ether (4×30 mL). These extracts were discarded. The aqueous phase was then basified (pH>12) by addition of 2.5N aqueous sodium hydroxyde solution. The crude product was extracted with ethyl acetate (4×30 mL). The combined organic layers were dried over MgSO₄ and concentrated under reduced pressure to afford a slightly yellow oil which became colorless after purification by Kugelrohr distillation (190° C.) in 68% yield.

IR (neat): 3322, 2944, 2802, 1721, 1612, 1457, 1281, 1122, 1012-¹H NMR (CDCl₃) δ=2.27 (s, 3H, NCH₃); 2.44 (m, 8H, 2×NCH₂CH₂N); 3.53 (s, 2H, ArCH₂N); 3.88 (s, 3H, OCH₃); 7.40 (d, 2H, J=8.3 Hz, 2×ArH); 7.91 (d, 2H, J=8.3 Hz, 2×ArH)-¹³C NMR (CDCl₃) δ=45.8 (NCH₃); 51.8 (OCH₃); 52.9 (2×CH₂N); 54.9 (2×CH₂N); 62.4 (ArCH₂N); 128.7 (2×ArC); 129.3 (2×ArC); 143.7 (ArC); 166.7 (ArCO₂CH₃)-MS CI (m/z) (%): 249 (M+1, 100%).

### 2-Methyl-5-tert-butoxycarbonylamino-aniline

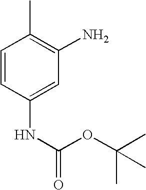

A solution of di-tert-butyldicarbonate (70 g, 320 mmol) in methanol (200 mL) was added over 2 h to a cold (−10° C.) solution of 2,4-diaminotoluene (30 g, 245 mmol) and triethylamine (30 mL) in methanol (15 mL). The reaction was followed by thin layer chromatography (hexane/ethyl acetate, 3:1) and stopped after 4h by adding 50 mL of water. The mixture was concentrated in vacuo and the residue was dissolved in 500 mL of ethyl acetate. This organic phase was washed with water (1×150 mL) and brine (2×150 mL), dried over MgSO₄, and concentrated under reduced pressure. The resulting light brown solid was washed with small amounts of diethyl ether to give off-white crystals of 2-methyl-5-tert-butoxycarbonylamino-aniline in 67% yield.

IR (neat): 3359; 3246; 2970; 1719; 1609; 1557; 1173; 1050 cm⁻¹-¹H NMR (CDCl₃): δ=1.50 (s, 9H, tBu); 2.10 (s, 3H, ArCH₃); 3.61 (br s, 2H, NH₂); 6.36 (br s, 1H, NH); 6.51 (dd, 1H, J=7.9 Hz, 2.3 Hz, ArH); 6.92 (d, 1H, J=7.9 Hz, ArH); 6.95 (s, 1H, ArH)-¹³C NMR (CDCl₃) δ=16.6 (ArCH₃); 28.3 (C(CH₃)₃); 80.0 (C(CH₃)₃); 105.2 (ArC); 108.6 (ArC); 116.9 (ArC); 130.4 (ArC—CH₃); 137.2 (ArC—N); 145.0 (ArC—NH₂); 152.8 (COOtBu) MS ESI (m/z) (%): 223 (M+1), 167 (55, 100%).

### N-(2-methyl-5-tert-butoxycarbonylamino)phenyl-thiourea

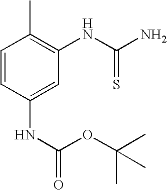

Benzoyl chloride (5.64 g, 80 mmol) was added dropwise to a well-stirred solution of ammonium thiocyanate (3.54 g, 88 mmol) in acetone (50 mL). The mixture was refluxed for 15 min, then, the hydrobromide salt of 2-methyl-5-tert-butoxycarbonylamino-aniline (8.4 g, 80 mmol) was added slowly portionswise. After 1 h, the reaction mixture was poured into ice-water (350 mL) and the bright yellow precipitate was isolated by filtration. This crude solid was then refluxed for 45 min in 70 mL of 2.5 N sodium hydroxide solution. The mixture was cooled down and basified with ammonium hydroxide. The precipitate of crude thiourea was recovered by filtration and dissolved in 150 mL of ethyl acetate. The organic phase was washed with brine, dried over Na₂SO₄, and concentrated under reduced pressure. The residue was purified by column chromatography (hexane/ethyl acetate, 1:1) to afford 63% of N-(2-methyl-5-tert-butoxycarbonylamino)phenyl-thiourea as a white solid.

IR (neat): 3437, 3292, 3175, 2983, 1724, 1616, 1522, 1161, 1053 cm⁻¹-¹H NMR (DMSO-⁶) δ=1.46 (s, 9H, tBu); 2.10 (s, 3H, ArCH₃); 3.60 (br s, 2H, NH₂); 7.10 (d, 1H, J=8.29 Hz, ArH); 7.25 (d, 1H, J=2.23 Hz, ArH); 7.28 (d, 1H, J=2.63 Hz, ArH); 9.20 (s, 1H, ArNH); 9.31 (s, 1H, ArNH)-¹³C NMR (DMSO-d⁶) δ=25.1 (ArCH₃); 28.1 (C(CH₃)₃); 78.9 (C(CH₃)₃); 116.6 (ArC); 117.5 (ArC); 128.0 (ArC); 130.4 (ArC—CH₃); 136.5 (ArC—NH); 137.9 (ArC—NH); 152.7 (COOtBu); 181.4 (C═S)—MS CI(m/z): 282 (M+1, 100%); 248 (33); 226 (55); 182 (99); 148 (133); 93 (188).

### 2-(2-methyl-5-tert-butoxycarbonylamino)phenyl-4-(3-pyridyl)-thiazole

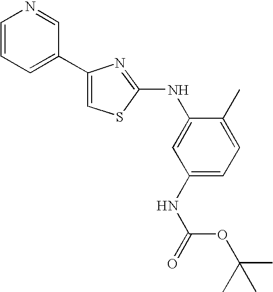

A mixture of 3-bromoacetyl-pyridine, HBr salt (0.81 g, 2.85 mmol), N-(2-methyl-5-tert-butoxycarbonylamino)phenyl-thiourea (0.8g, 2.85 mmol) and KHCO₃ (˜0.4 g) in ethanol (40 mL) was heated at 75° C. for 20 h. The mixture was cooled, filtered (removal of KHCO₃) and evaporated under reduced pressure. The residue was dissolved in CHCl₃ (40 mL) and washed with saturated aqueous sodium hydrogen carbonate solution and with water. The organic layer was dried over Na₂SO₄ and concentrated. Colum chromatographic purification of the residue (hexane/ethyl acetate, 1:1) gave the desired thiazole in 70% yield as an orange solid

IR (neat): 3380, 2985, 2942, 1748, 1447, 1374, 1239, 1047, 938-¹H NMR (CDCl₃) δ=1.53 (s, 9H, tBu); 2.28 (s, 3H, ArCH₃); 6.65 (s, 1H, thiazole-H); 6.89 (s, 1H); 6.99 (dd, 1H, J=8.3 Hz, 2.3 Hz); 7.12 (d, 2H, J=8.3 Hz); 7.35 (dd, 1H, J=2.6 Hz, 4.9 Hz); 8.03 (s, 1); 8.19 (dt, 1H, J=1.9 Hz, 7.9 Hz); 8.54 (br s, 1H, NH); 9.09 (s, 1H, NH)—¹³C NMR (CDCl₃) δ=18.02 (ArCH₃); 29.2 (C(CH₃)₃); 81.3 (C(CH₃)₃); 104.2 (thiazole-C); 111.6; 115.2; 123.9; 124.3; 131.4; 132.1; 134.4; 139.5; 148.2; 149.1; 149.3; 153.6; 167.3 (C═O)-MS CI (m/z) (%): 383 (M+1, 100%); 339 (43); 327 (55); 309 (73); 283 (99); 71 (311).

### 2-(2-methyl-5-amino)phenyl-4(3-pyridyl)-thiazole

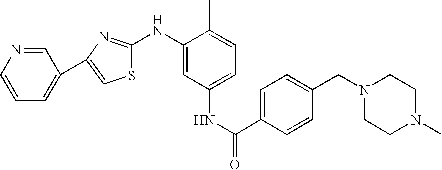

2-(2-methyl-5-tert-butoxycarbonylamino)phenyl-4-(3-pyridyl)-thiazole (0.40 g, 1.2 mmol) was dissolved in 10 mL of 20% TFA/CH₂Cl₂. The solution was stirred at room temperature for 2 h, then it was evaporated under reduced pressure. The residue was dissolved in ethyl acetate. The organic layer was washed with aqueous 1N sodium hydroxide solution, dried over MgSO₄, and concentrated to afford 2-(2-methyl-5-amino)phenyl-4-(3-pyridyl)-thiazole as a yellow-orange solid in 95% yield. This crude product was used directly in the next step.

A 2M solution of trimethyl aluminium in toluene ( 2.75 mL) was added dropwise to a cold (0° C.) solution of 2-(2-methyl-5-amino)phenyl-4-(3-pyridyl)-thiazole (0.42 g, 1.5 mmol) in anhydrous dichloromethane (10 mL) under argon atmosphere. The mixture was warmed to room temperature and stirred at room temperature for 30 min. A solution of methyl-4-(1-N-methyl-piperazino)-methyl benzoate (0.45 g, 1.8 mmol) in anhydrous. dichloromethane (1 mL) and added slowly, and the resulting mixture was heated at reflux for 5 h. The mixture was cooled to 0° C. and quenched by dropwise addition of a 4N aqueous sodium hydroxide solution (3 mL). The mixture was extracted with dichloromethane (3×20 mL). The combined organic layers were washed with brine (3×20 mL) and dried over anhydrous MgSO₄. (2-(2-methyl-5-amino)phenyl4-(3-pyridyl)-thiazole) is obtained in 72% after purification by column chromatography (dichloromethane/methanol, 3:1)

IR (neat): 3318, 2926, 1647, 1610, 1535, 1492, 1282, 1207, 1160, 1011, 843-¹HNMR (CDCl₃) δ=2.31 (br s, 6H, ArCH₃+NCH₃); 2.50 (br s, 8H, 2×NCH₂CH₂N); 3.56 (s, 2H, ArCH₂N); 6.89 (s, 1H, thiazoleH); 7.21-7.38 (m, 4H); 7.45 (m, 2H); 7.85 (d, 2H, J=8.3 Hz); 8.03 (s, 1H); 8.13 (s, 1H); 8.27 (s, 1H); 8.52 (br s, 1H); 9.09 (s, 1H, NH)—¹³C NMR (CDCl₃) δ=17.8 (ArCH₃); 46.2 (NCH₃); 53.3 (NCH₂); 55.3 (NCH₂); 62.8 (ArCH₂N); 99.9 (thiazole-C); 112.5; 123.9; 125.2; 127.5; 129.6; 131.6; 133.7; 134.0; 137.6; 139.3; 142.9; 148.8; 149.1; 166.2 (C═O); 166.7 (thiazoleC—NH)—MS CI (m/z) (%): 499 (M+H, 100%); 455 (43); 430 (68); 401 (97); 374 (124); 309 (189); 283 (215); 235 (263); 121 (377); 99 (399).

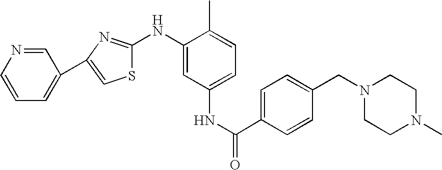

The expression “type II diabetes” as referred herein includes obesity, hypercholesterolemia, hypergycemia, hypertension, endothelial dysfunction, insulin resistance, and vascular remodelling.

In a further embodiment, c-kit inhibitors as mentioned above are inhibitors of activated c-kit. In frame with the invention, the expression “activated c-kit” means a constitutively activated-mutant c-kit including at least one mutation selected from point mutations, deletions, insertions, but also modifications and alterations of the natural c-kit sequence (SEQ ID No1). Such mutations, deletions, insertions, modifications and alterations can occur in the transphosphorylase domain, in the juxtamembrane domain as well as in any domain directly or indirectly responsible for c-kit activity. The expression “activated c-kit” also means herein SCF-activated c-kit. Preferred and optimal SCF concentrations for activating c-kit are comprised between 5.10⁻⁷ M and 5.10⁻⁶ M, preferably around 2.10⁻⁶ M. In a preferred embodiment, the activated-mutant c-kit in step a) has at least one mutation proximal to Y823, more particularly between amino acids 800 to 850 of SEQ ID No1 involved in c-kit autophosphorylation, notably the D816V, D816Y, D816F and D820G mutants. In another preferred embodiment, the activated-mutant c-kit in step a) has a deletion in the juxtamembrane domain of c-kit. Such a deletion is for example between codon 573 and 579 called c-kit d(573-579). The point mutation V559G proximal to the juxtamembrane domain c-kit is also of interest.

In this regard, the invention contemplates a method for treating type II diabetes as defined above comprising administering to a human in need of such treatment a compound that is a selective, potent and non toxic inhibitor of activated c-kit obtainable by a screening method which comprises:
a) bringing into contact (i) activated c-kit and (ii) at least one compound to be tested; under conditions allowing the components (i) and (ii) to form a complex,
b) selecting compounds that inhibit activated c-kit,
c) testing and selecting a subset of compounds identified in step b), which are unable to promote death of IL-3 dependent cells cultured in presence of IL-3.

This screening method can further comprise the step consisting of testing and selecting a subset of compounds identified in step b) that are inhibitors of mutant activated c-kit (for example in the transphosphorylase domain), which are also capable of inhibiting SCF-activated c-kit wild.

Alternatively, in step a) activated c-kit is SCF-activated c-kit wild.

A best mode for practicing this method consists of testing putative inhibitors at a concentration above 10 μM in step a). Relevant concentrations are for example 10, 15, 20, 25, 30, 35 or 40 μM.

In step c), IL-3 is preferably present in the culture media of IL-3 dependent cells at a concentration comprised between 0.5 and 10 ng/ml, preferably between 1 to 5 ng/ml.

Examples of IL-3 dependent cells include but are not limited to:
cell lines naturally expressing and depending on c-kit for growth and survival. Among such cells, human mast cell lines can be established using the following procedures: normal human mast cells can be infected by retroviral vectors containing sequences coding for a mutant c-kit comprising the c-kit signal peptide and a TAG sequence allowing to differentiate mutant c-kits from c-kit wild expressed in hematopoetic cells by means of antibodies.

This technique is advantageous because it does not induce cellular mortality and the genetic transfer is stable and gives satisfactory yields (around 20%). Pure normal human mast cells can be routinely obtained by culturing precursor cells originating from blood obtained from human umbilical vein. In this regard, heparinated blood from umbilical vein is centrifuged on a Ficoll gradient so as to isolate mononucleated cells from other blood components. CD34+ precursor cells are then purified from the isolated cells mentioned above using the immunomagnetic selection system MACS (Miltenyi biotech). CD34+ cells are then cultured at 37° C. in 5% CO₂ atmosphere at a concentration of 10⁵ cells per ml in the medium MCCM (α-MEM supplemented with L-glutamine, penicillin, streptomycin, 5 10⁻⁵ M β-mercaptoethanol, 20% veal fœtal serum, 1% bovine albumin serum and 100 ng/ml recombinant human SCF. The medium is changed every 5 to 7 days. The percentage of mast cells present in the culture is assessed each week, using May-Grünwal Giemsa or Toluidine blue coloration. Anti-tryptase antibodies can also be used to detect mast cells in culture. After 10 weeks of culture, a pure cellular population of mast cells (>98%) is obtained.

It is possible using standard procedures to prepare vectors expressing c-kit for transfecting the cell lines established as mentioned above. The cDNA of human c-kit has been described in Yarden et al., (1987) EMBO J.6 (11), 3341-3351. The coding part of c-kit (3000 bp) can be amplified by PCR and cloned, using the following oligonucleotides:

(SEQ ID No2)

| | |
|---|---|
| 5′AAGAAGAGATGGTACCTCGAGGGGTGACCC3′ | sens |

(SEQ ID No3)

| | |
|---|---|
| 5′CTGCTTCGCGGCCGCGTTAACTCTTCTCAACCA3′ | antisens |

The PCR products, digested with Not1 and Xho1, has been inserted using T4 ligase in the pFlag-CMV vector (SIGMA), which vector is digested with Not1 and Xho1 and dephosphorylated using CIP (Biolabs). The pFlag-CMV-c-kit is used to transform bacterial clone XL1-blue. The transformation of clones is verified using the following primers:

(SEQ ID No4)

| | |
|---|---|
| 5′AGCTCGTTTAGTGAACCGTC3′ | sens, |

(SEQ ID No5)

| | |
|---|---|
| 5′GTCAGACAAAATGATGCAAC3′ | antisens. |

Directed mutagenesis is performed using relevant cassettes is performed with routine and common procedure known in the art.

The vector Migr-1 (ABC) can be used as a basis for constructing retroviral vectors used for transfecting mature mast cells. This vector is advantageous because it contains the sequence coding for GFP at the 3′ and of an IRES. These features allow to select cells infected by the retrovirus using direct analysis with a fluorocytometer. As mentioned above, the N-terminal sequence of c-kit c-DNA can be modified so as to introduce a Flag sequence that will be useful to discriminating heterogeneous from endogenous c-kit

Other IL-3 dependent cell lines that can be used include but are not limited to:
BaF3 mouse cells expressing wild-type or mutated form of c-kit (in the juxtamembrane and in the catalytic sites) are described in Kitayama et al, (1996), Blood 88, 995-1004 and Tsujimura et al, (1999), Blood 93, 1319-1329.
IC-2 mouse cells expressing either c-kit^{WT} or c-kit^{D814Y} are presented in Piao et al, (1996), Proc. Natl. Acad. Sci. USA 93, 14665-14669.

IL-3 independent cell lines are:
HMC-1, a factor-independent cell line derived from a patient with mast cell leukemia, expresses a juxtamembrane mutant c-kit polypeptide that has constitutive kinase activity (Furitsu T et al, J Clin Invest. 1993; 92:1736-1744; Butterfield et al, Establishment of an immature mast cell line from a patient with mast cell leukemia Leuk Res. 1988; 12:345-355 and Nagata et al, Proc Natl Acad Sci U S A. 1995; 92:10560-10564).
P815 cell line (mastocytoma naturally expressing c-kit mutation at the 814 position) has been described in Tsujimura et al, (1994), Blood 83, 2619-2626.

The extent to which component (ii) inhibits activated c-kit can be measured in vitro or in vivo. In case it is measured in vivo, cell lines expressing an activated-mutant c-kit, which has at least one mutation proximal to Y823, more particularly between amino acids 800 to 850 of SEQ ID No1 involved in c-kit autophosphorylation, notably the D816V, D816Y, D816F and D820G mutants, are preferred.

Example of cell lines expressing an activated-mutant c-kit are as mentioned above.

In another preferred embodiment, the method further comprises the step consisting of testing and selecting compounds capable of inhibiting c-kit wild at concentration below 1 μM. This can be measured in vitro or in vivo.

Therefore, compounds are identified and selected according to the method described above are potent, selective and non-toxic c-kit wild inhibitors.

Alternatively, the screening method as defined above can be practiced in vitro. In this regard, the inhibition of mutant-activated c-kit and/or c-kit wild can be measured using standard biochemical techniques such as immunoprecipitation and western blot. Preferably, the amount of c-kit phosphorylation is measured.

In a still further embodiment, the invention contemplates a method for treating type II diabetes as depicted above wherein the screening comprises:
a) performing a proliferation assay with cells expressing a mutant c-kit (for example in the transphosphorylase domain), which mutant is a permanent activated c-kit, with a plurality of test compounds to identify a subset of candidate compounds targeting activated c-kit, each having an IC50<10 μM, by measuring the extent of cell death,
b) performing a proliferation assay with cells expressing c-kit wild said subset of candidate compounds identified in step (a), said cells being IL-3 dependent cells cultured in presence of IL-3, to identify a subset of candidate compounds targeting specifically c-kit,
c) performing a proliferation assay with cells expressing c-kit, with the subset of compounds identified in step b) and selecting a subset of candidate compounds targeting c-kit wild, each having an IC50<10 μM, preferably an IC50<1 μM, by measuring the extent of cell death.

Here, the extent of cell death can be measured by 3H thymidine incorporation, the trypan blue exclusion method or flow cytometry with propidium iodide. These are common techniques routinely practiced in the art.

The method according to the invention includes preventing, delaying the onset and/or treating type II diabetes and associated damages in humans.

In the method defined above, any compound capable of depleting mast cells can be used. Such compounds can belong to, as explicated above, tyrosine kinase inhibitors, such as c-kit inhibitors, but are not limited to any particular family so long as said compound shows capabilities to deplete mast cells. Depletion of mast cells can be evaluated using for example one of the mast cell lines depicted above using routine procedure.

Best compounds are compounds exhibiting the greatest selectivity.

Control cell lines include other hematopoeitic cells that are not mast cells or related cells or cell lines. These control cell lines include SCF independent expanded human CD34+ normal cells. These control cells also include but are not limited to the human T lymphocyte Jurkat cell line (ATCC No TIB-152 and mutant cell lines derived thereof), the human B lymphocyte Daudi or Raji cell line (ATCC No CCL-213 and CCL-86 respectively), the human monocytic U 937 cell line (ATCC No CRL-1593.2) and the human HL-60 cell line (ATCC No CCL-240) and mutant cell lines derived thereof CRL-2258 and CRL-2392).

Such compounds can be selected with a method for identifying compounds capable of depleting mast cells, said compound being non-toxic for cell types other than mast cells, comprising the step consisting of:
a) culturing mast cells in vitro in a culture medium suitable for mast cells,
b) adding to said culture medium at least one compound to be tested and incubating said cells for a prolonged period of time,
c) selecting compounds that promote mast cells death,
d) identifying a subset of compounds selected in step c) that are unable to promote death of cells selected from the above mentioned control cell lines.

Therefore, the invention embraces the use of the compounds defined above to manufacture a medicament for treating type II diabetes including obesity, hypercholesterolemia, hypergycemia, hypertension, endothelial dysfunction, insulin resistance, and vascular remodelling.

More particularly, the above compounds are useful for preventing the onset or development of type II diabetes in obese persons.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

More particularly, the invention relates to a pharmaceutical composition intended for oral administration.

Pharmaceutical compositions suitable for use in the invention include compositions wherein compounds for depleting mast cells, such as c-kit inhibitors, or compounds inhibiting mast cells degranulation are contained in an effective amount to achieve the intended purpose. The determination of an effective dose is well within the capability of those skilled in the art. A therapeutically effective dose refers to that amount of active ingredient, which ameliorates the symptoms or condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions which exhibit large therapeutic indices are preferred.

### Example 1

Effect of different c-kit inhibitors on serum glucose, insulin, triglycerides, cholesterol and non esterified fatty acids levels in db/db mice

1.1 Purpose of this Study

The objective of this study is to assess the effects of different c-kit inhibitors on serum glucose, insulin, triglycerides, cholesterol and non esterified fatty acids levels in male db/db mice dosed orally, once-a-day, for 5 days.

1.2 Materials and Methods

1.2.2 Test System

30 male 9/11 weeks old C57BL/Ks J Rj-db (db/db) mice (Janvier, France or Harlan, France), weighing in the target range of 30 to 50 g, will be included in this study. They will be housed in a temperature (19.5-24.5° C.) and relative humidity (45-65%) controlled room with a 12-h light/dark cycle, with ad libitum access to filtered tap-water and irradiated pelleted laboratory chow (ref. A04, U.A.R., France) throughout the study.

Upon receipt at animal facilities, they will be housed 5 per cage and at least a 5-day acclimatization period will be observed. Animals will be individually identified on the tail.

1.2.3 Study Materials

Substance tested code: substances No1 and No2
Reference substance
Code: rosiglitazone 10 mg/kg
Source: Sequoia Research Products Ltd, UK
   Vehicle

The vehicle will be defined by the Sponsor but, if no indication is supplied, a 3% Arabic gum aqueous solution (w/v) will be used.
Reagents
Glucose kit (ref. 442640, Beckman Coulter, France)
Insulin ELIT Plus kit (ref. INSRAT01-8N, Eurobio, France)
Triglycerides kit (ref. 445850, Beckman Coulter, France)
Total cholesterol kit (ref. 467825, Beckman Coulter, France)
Non esterified fatty acids kit (NEFA, ref. FA115, Randox, France)
Isoflurane (Forene®, Abbott, UK)
Principal Equipment
Balances (AT261 model, Mettler, France)
Centrifuge (2K15 model, Sigma, France)
Multi-parametric analyzer (Synchron CX-4 model, Beclanan Coulter, France)
Microplate reader (Multiskan RC model, Labsystem, France)
Principal Data Processing Systems
Excel (Microsoft v.2000), Biolise (Labsystem v.2.65) and SigmaStat (SPSS v.2.03)
   1.2.4 Study Design

6 groups of 5 animals each will be included in this study:
Group 1: vehicle
Group 2: test substance No1, dose 1, route of administration
Group 3: test substance No1, dose 2, route of administration
Group 4: test substance No2, dose 1, route of administration
Group 5: test substance No2, dose 2, route of administration
Group 6: rosiglitazone, 10 mg/kg, route of administration

The doses will be expressed in terms of free active substance.

The test and reference substances will be extemporaneously prepared as instructed.

The test and reference substances and the vehicle will be administered in a volume of 5 ml/kg adjusted according to individual body weight values.

1.2.5 Experimental Protocol

One to three days before beginning the treatments (T0), mice will be weighed and blood samples will be collected through the retro-orbital plexus under isoflurane anesthesia. Blood samples will be kept at room temperature for 5 to 10 min to form a spontaneous clot, then put in ice until they are centrifuged at 3500×g for 10-15 min at 4° C. An aliquot of serum will be used for measuring glucose levels.

Six groups of 5 mice will be formed with respect to homogeneous glycemia values by using a randomization table. Animal showing glycemia below 20 mM will be excluded from the study.

From T1 to T5, the mice will be weighed and dosed once daily for 5 consecutive days at constant time.

At T5, 2 hours after the last administration, blood samples will be collected through the retro-orbital plexus under isoflurane anesthesia. Blood samples will be kept at room temperature for 5 to 10 min to form a spontaneous clot, then put in ice until they are centrifuged at 3500×g for 10 min at 4° C. Serum will be aliquoted and frozen at −20° C. until use. After blood collection, the mice will be euthanized by cervical dislocation.

Serum glucose and triglycerides levels will be determined using the Synchron C×4 analyzer. Serum non esterified fatty acids levels will be measured manually using a colorimetric method, and insulin levels determined by ELISA.

1.2.6 Analysis and Expression of Results

The results will be expressed as mean±SEM:
Glucose levels (mM) at T5
Insulin levels (nM) at T5
Triglycerides levels (mM) at T5
Non esterified fatty acids levels (mM) at T5

For each parameter, a % of effect will be calculated according to following formula:
((vehicle group−test group)/vehicle group)*100

Statistical analysis will consist in a one-way analysis of variance followed by multiple comparisons versus the vehicle group (Dunnett's test). In case the equal variance test fails, a Kruskall-Wallis one-way analysis of variance on ranks will be proposed. A difference will be considered significant for p<0.05.

This study has been performed according to the model described in Grasa et al. , Oleoyl-estrone lowers the body weight of ob/ob and db/db mice. Horm. Metab. Res. 2000, 32, 246-250 and has be subjected to Quality Assurance monitoring.

3. RESULTS: Effects of AB Compounds on Serum Biomarkers in Male db/db Mice Dosed P.O. for 5 Days

TABLE 1

| | | N^{o}1 | N^{o}1 |
|---|---|---|---|
| Treatment | Vehicle | 100 | 200 |
| (mg/kg) | (n = 5) | (n = 5) | (n = 5) |
| Glucose | 27.46 ± 2.95 | 21.90 ± 3.01 | 19.72 ± 1.23 |
| (mM) | | | |
| Effect (%) | | 20 | 28 |
| Cholesterol | 3.38 ± 0.08 | 3.37 ± 0.12 | 2.79 ± 0.29 |
| (mM) | | | |
| Effect (%) | | 0 | 17 |
| Triglycerides | 1.57 ± 0.11 | 1.00 ± 0.08** | 0.89 ± 0.06** |
| (mM) | | | |
| Effect (%) | | 36 | 43 |
| NEFA | 0.82 ± 0.05 | 0.68 ± 0.14 | 0.79 ± 0.084 |
| (mM) | | | |
| Effect (%) | | 17 | 3 |
| Insulin | 3.69 ± 0.86 | 3.31 ± 0.89 | 2.06 ± 0.38 |
| (nM) | | | |
| Effect (%) | | 10 | 44 |

| | N^{o}2 | N^{o}2 | Rosiglitazone | |
|---|---|---|---|---|
| Treatment | 100 | 200 | 10 | ANOVA |
| (mg/kg) | (n = 5) | (n = 5) | (n = 5) | (P value) |
| Glucose (mM) | 18.16 ± 2.22 | 15.64 ± 3.23* | 11.25 ± 2.65** | 0.006 |
| Effect (%) | 34 | 43 | 59 | |
| Cholesterol (mM) | 3.24 ± 0.09 | 2.72 ± 0.13 | 2.74 ± 0.47 | 0.163 |
| Effect (%) | 4 | 19 | 19 | |
| Triglycerides | 0.77 ± 0.08** | 0.66 ± 0.05** | 0.46 ± 0.10** | <0.001 |
| (mM) | | | | |
| Effect (%) | 51 | 58 | 71 | |
| NEFA | 0.70 ± 0.048 | 0.71 ± 0.07 | 0.31 ± 0.061* | 0.036 (°) |
| (mM) | | | | |
| Effect (%) | 14 | 13 | 63 | |
| Insulin | 2.15 ± 0.42 | 2.82 ± 0.59 | 0.82 ± 0.27* | 0.039 |
| (nM) | | | | |
| Effect (%) | 42 | 24 | 77 | |

Values are expressed as mean ± SEM
Statistics: One-way analysis of variance (ANOVA) followed by a Dunnett's test. or (°) Kruskal Wallis analysis of variance on the ranks followed by a Dunn's test.
*p < 0.05,
**p < 0.01 as compared to vehicle
Effect % as compared to vehicle

### Example 2

in vitro TK Inhibition Assays

Procedure

Experiments were performed using purified intracellular domain of c-kit expressed in baculovirus. Estimation of the kinase activity was assessed by the phosphoiylation of tyrosine containing target peptide estimated by established ELISA assay.

Experimental Results on Tested Compounds

Result in Table 2 shows the potent inhibitory action of the catalytic activity of c-kit with an IC50<10 μM. Further experiments (not shown) indicates that at least one compound acts as perfect competitive inhibitors of ATP.

TABLE 2

| | In vitro Inhibition assay |
|---|---|
| | results c-kit |
| Compounds | IC50 (μM) |
| 066; 074; 078; 084; 012; 016; 073; 021; 088; | <10 μM |
| 023; 025; 047; 048; 055; 049; 026; 087; 075; | |
| 089; 051; 082; 090; 060; 085; 052; 053; 096 | |

### Example 2

ex vivo TK Inhibition Assays

Procedures

C-Kit WT and mutated C-Kit (JM) assay

Proliferation Assays

Cells were washed two times in PBS before plating at 5×104 cells per well of 96-well plates in triplicate and stimulated either with hematopoietic growth factors (HGF) or without. After 2 days of culture, 37 Bq (1.78 Tbq/mmol) of [3H] thymidine (Amersham Life Science, UK) was added for 6 hours. Cells were harvested and filtered through glass fiber filters and [3H] thymidine incorporation was measured in a scintillation counter. For proliferation assay, all drugs were prepared as 2OmM stock solutions in DMSO and conserved at −80° C. Fresh dilutions in PBS were made before each experiment DMSO dissolved drugs were added at the beginning of the culture. Control cultures were done with corresponding DMSO dilutions. Results are represented in percentage by taking the proliferation without inhibitor as 100%.

Cells

Ba/F3 murine kit and human kit, Ba/F3 mkitΔ27 (juxamembrane deletion) are derived from the murine IL-3dependent Ba/F3 proB lymphoid cells. The FMA3 and P815 cell lines are mastocytoma cells expressing endogenous mutated forms of Kit, i.e., frame deletion in the murine juxtamembrane coding region of the receptor-codons 573 to 579.

The Human Leukaemic MC Line HMC-1 Expresses Mutations JM-V560G; Immunoprecipitation Assays and Western Blotting Analysis

For each assay, 5.10₆ Ba/F3 cells and Ba/F3-derived cells with various c-kit mutations were lysed and immunoprecipitated as described (Beslu et al., 1996), excepted that cells were stimulated with 250 ng / ml of rmKL. Cell lysates were immunoprecipitated with a rabbit immunserum anti murine KIT, directed against the KIT cytoplasmic domain (Rottapel et al., 1991). Western blot was hybridized either with the 4G10 anti-phosphotyrosine antibody (UBI) or with the rabbit immunserum anti-murine KIT or with different antibodies (described in antibodies paragraph). The membrane was then incubated either with HRP-conjugated goat anti mouse IgG antibody or with HRP-conjugated goat anti rabbit IgG antibody (Immunotech), Proteins of interest were then visualized by incubation with ECL reagent (Amersham).

Experimental Results

The experimental results for various compounds according to the invention using above-described protocols are set forth at Table 3:

TABLE 3

| Target | IC50 (μM) | Compounds |
|---|---|---|
| c-Kit WT | IC50 < 10 μM | 002; 005; 006; 007; 008; 009; 010; 012; 017; 019; 020; |
| | | 021; 023; 024; 025; 026; 028; 029; 030; 032; 042; 043; |
| | | 045; 047; 048; 049; 050; 051; 052; 053; 054; 055; 056; |
| | | 057; 059; 060; 061; 062; 063; 064; 065; 066; 067; 072; |
| | | 073; 074; 075; 077; 078; 079; 080; 081; 082; 083; 084; |
| | | 085; 086; 087; 088; 089; 090; 092; 093; 094; 095; 096; |
| | | 097; 106; 105; 104; 103; 128; 129; 130; 131; 117; 110; |
| | | 116; 124; 108; 122; 111; 113; 118; 107; |
| c-Kit JM Δ27 | IC50 < 1 μM | 028; 074; 029; 009; 012; 073; 020; 042; 061; 065; 088; |
| | | 025; 048; 049; 050; 089; 051; 082; 090; 083; 059; 052; |
| | | 053; 066; 103; 067; 104; 078; 079; 105; 081; 084; 030; |
| | | 010; 021; 043; 054; 062; 106; 023; 024; 064; 047; 055; |
| | | 026; 087; 075; 085; 005; 077; 092; 060; 032; 017; 063; |
| | | 093; 094; 095; 086; 093; 096; 108; 117; 122; 008; 080; |
| | | 111; 118; 113; 007; 072; 019; 056; 057; 107; 097; |

1. A method for treating type II diabetes, obesity and related disorders comprising administering a compound capable of depleting mast cells or a compound inhibiting mast cells degranulation in a human in need of such treatment.

2. A method according to claim 1 for treating type II diabetes comprising administering a c-kit inhibitor to a human in need of such treatment.

3. A method according to claim 2, wherein said c-kit inhibitor is a non- toxic, selective and potent c-kit inhibitor wherein it is unable to promote death of IL-3 dependent cells cultured in presence of IL-3.

4. A method according claim 1 wherein said inhibitor is selected from the group consisting of:
2-(3-amino) arylamino-4-aryl-thiazoles,
pyrimidine derivatives, more particularly N-phenyl-2-pyrimidine-amine derivatives,
indolinone derivatives, more particularly pyrrol-substituted indolinones,
monocyclic, bicyclic aryl and heteroaryl compounds,
and quinazoline derivatives.

5. A method according to claim 4, wherein said inhibitor is selected from the group consisting of N-phenyl-2-pyrimidine-amine derivatives having the formula II: 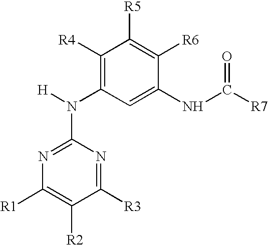
Wherein R2 and R3 are independently chosen from H, F, Cl, Br, I, a C1-C5 alkyl or a cyclic or heterocyclic group, especially a pyridyl group;
R4, R5 and R6 are independently chosen from H, F, Cl, Br, I, a C1-C5 alkyl, especially a methyl group;
and R7 is a phenyl group bearing at least one substituent, which in turn possesses at least one basic site, such as an amino function, preferably the following group:

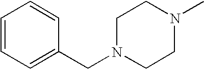

6. A method according to claim 5, wherein said inhibitor is the 4-(4-méthylpipérazine-1-ylméthyl)-N-[4-méthyl-3-(4-pyridine-3 -yl)pyrimidine-2 ylamino)phényl]-benzamide.

7. A method according to claim 3, wherein said c-kit inhibitor is an inhibitor of activated c-kit.

8. A method according to claim 7, wherein said inhibitor is capable of inhibiting constitutively activated-mutant c-kit.

9. A method according to claim 7, wherein said activated c-kit inhibitor is capable of inhibiting SCF-activated c-kit.

10. A method according to claim 4, wherein said c-kit inhibitor is selected from compounds belonging to the 2-(3-amino)arylamino-4-aryl-thiazoles of formula III: 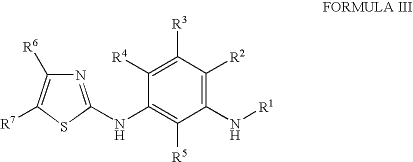
and wherein R¹ is:
a) a linear or branched alkyl group containing from 1 to 10 carbon atoms optionally substituted with at least one heteroatom, notably a halogen selected from I, Cl, Br and F, and/or bearing a pendant basic nitrogen functionality;
b) an aryl or heteroaryl group optionally substituted by an alkyl or aryl group optionally substituted with a heteroatom, notably a halogen selected from I, Cl, Br and F or bearing a pendant basic nitrogen functionality;
c) a-CO—NH—R, —CO—R,—CO—OR or a-CO—NRR′ group, wherein R and R′ are independently chosen from H or an aryl, heteroaryl, alkyl and cycloalkyl group optionally substituted with at least one heteroatom, notably a halogen selected from I, Cl, Br and F, and/or bearing a pendant basic nitrogen functionality;
R² is hydrogen, halogen or a linear or branched alkyl group containing from 1 to 10 carbon atoms, trifluoromethyl or alkoxy;
R³ is hydrogen, halogen or a linear or branched alkyl group containing from 1 to 10 carbon atoms, trifluoromethyl or alkoxy;
R⁴ is hydrogen, halogen or a linear or branched alkyl group containing from 1 to 10 carbon atoms, trifluoromethyl or alkoxy;
R⁵ is hydrogen, halogen or a linear or branched alkyl group containing from 1 to 10 carbon atoms, trifluoromethyl or alkoxy;
R⁶ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy,
iv) H, a halogen selected from I, F, Cl or Br; NH₂, NO₂ or SO₂—R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality;
and R⁷ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy,
iv) H, a halogen selected from I, F, Cl or Br; NH₂, NO₂ or SO₂—R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality.

11. A method according to claim 10, wherein said c-kit inhibitor is selected from compounds belonging to the 2-(3-amino)arylamino-4-aryl-thiazoles of formula IV: 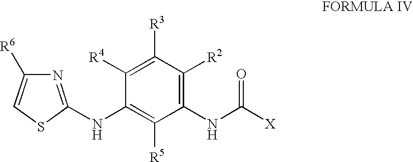
wherein X is R or NRR′ and wherein R and R′ are independently chosen from H, an aryl, an heteroaryl, an alkyl and a cycloalkyl group optionally substituted with at least one heteroatom, such as for example a halogen chosen from F, I, Cl and Br and optionally bearing a pendant basic nitrogen functionality; or an aryl, an heteroaryl, an alkyl and a cycloalkyl group substituted with an aryl, an heteroaryl, an alkyl and a cycloalkyl group optionally substituted with at least one heteroatom, such as for example a halogen chosen from F, I, Cl and Br and optionally bearing a pendant basic nitrogen functionality,
R² is hydrogen, halogen or a linear or branched alkyl group containing from 1 to 10 carbon atoms, trifluoromethyl or alkoxy;
R³ is hydrogen, halogen or a linear or branched alkyl group containing from 1 to 10 carbon atoms, trifluoromethyl or alkoxy;
R⁴ is hydrogen, halogen or a linear or branched alkyl group containing from 1 to 10 carbon atoms, trifluoromethyl or alkoxy;
R⁵ is hydrogen, halogen or a linear or branched alkyl group containing from 1 to 10 carbon atoms, trifluoromethyl or alkoxy;
R⁶ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.

12. A method according to claim 11, wherein X is a substituted alkyl, aryl or heteroaryl group bearing a pendant basic nitrogen functionality represented for example by the structures a to f shown below, wherein the wavy line corresponds to the point of attachment to core structure of formula IV:

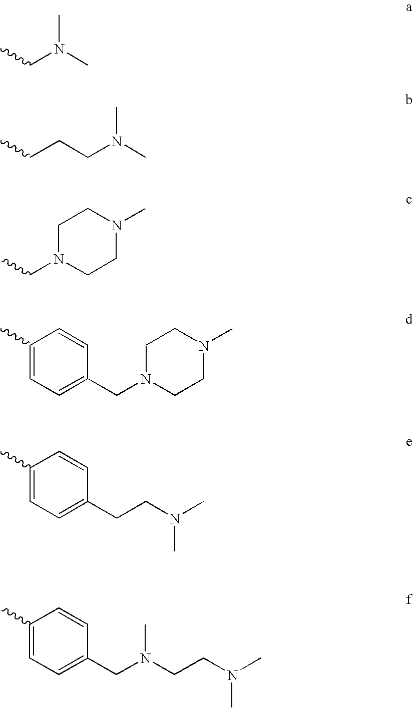

13. A method according to claim 12, wherein said c-kit inhibitor is selected from:
4-Diethylaminomethyl-N-[4-methyl-3-(4-pyridin-3-yl-thiazol-2-ylamino) -phenyl]-benzamide,
N-[4-Methyl-3-(4-pyridin-3-yl-thiazol-2-ylamino)-phenyl]-4-morpholin -4-ylmethyl-benzamide,
4-Dipropylaminomethyl-N-[4-methyl-3-(4-pyridin-3-yl-thiazol-2-ylamino) -phenyl]-benzamide,
N-[4-Methyl-3-(4-pyridin-3-yl-thiazol-2-ylamino)-phenyl]-4-piperidin-1-ylmethyl-benzamide,
3-Iodo-N-[4-methyl-3-(4-pyridine-3-yl-thiazol-2-ylamino)-phenyl]-benzamide,
4-Hydroxymethyl-N-[4-methyl-3-(4-pyridin-3-yl-thiazol-2-ylamino)-phenyl]-benzamide
4-{[4-Methyl-3(4-pyridin-3-yl-thiazol-2-ylamino)-phenylamino]-methyl}-benzoic acid methyl ester,
3-Phenyl-propynoic acid [4-methyl-3-(4-pyridin-3-yl-thiazol-2-ylamino)-phenyl]-amide,
4-Amino-N-[4-methyl-3-(4-pyridin-3-yl-thiazol-2-ylamino)-phenyl]-benzamide,
2-Iodo-N-[4-methyl-3-(4-pyridin-3-yl-thiazol-2-ylamino)-phenyl]-benzamide,
4-Iodo-N-[4-methyl-3-(4-pyridin-3-yl-thiazol-2-ylamino)-phenyl]-benzamide,
4-(3-{4-[4-Methyl-3-(4-pyridin-3-yl-thiazol-2-ylamino)-phenylcarbamoyl]-phenyl}-ureido)-benzoic acid ethyl ester,
N-[4-Methyl-3-(4-pyridin-3-yl-thiazol-2-ylamino)-phenyl]-4-[3-(4-trifluoromethyl -phenyl)-ureido]-benzamide,
4-[3-(4-Bromo-phenyl)-ureido]-N-[4-methyl-3-(4-pyridin-3-yl-thiazol-2ylamino)-phenyl]-benzamide,
{4-[4-Methyl-3-(4-pyridin-3-yl-thiazol-2-ylamino)-phenylcarbamoyl]-benzyl}carbamic acid tert-butyl ester,
4-Hydroxy-N-[4-methyl-3-(4-pyridin-3-yl-thiazol-2-ylamino)-phenyl]-benzamide
4-[(Diisopropylamino)-methyl]-N-[4-methyl-3-(4-pyridin-3-yl-thiazol-2-ylamino)-phenyl]-benzamide,
N-[4-Methyl-3-(4-pyridin-3-yl-thiazol-2-ylamino)-phenyl]-4-(3-thiophen-2-yl-ureido)-benzamide,
4-[3-(3,5-Dimethyl-isoxazol-4-yl)-ureido]-N-[4-methyl-3-(4-pyridin-3-yl-thiazol-2-ylamino)-phenyl]-benzamide,
4-[3-(4-Methoxy-phenyl)-ureido]-N-[4-methyl-3-(4-pyridin-3-yl-thiazol-2-ylamino)-phenyl]-benzamide,
4-[3-(4-Difluoromethoxy-phenyl)-ureido]-N-[4-methyl-3-(4-pyridin-3-yl-thiazol-2-ylamino)-phenyl]-benzamide,
Thiophene-2-sulfonic acid 4-[4-methyl-3-(4-pyridin-3-yl-thiazol-2-ylamino) phenylcarbamoyl]-phenyl ester,
4-lodo-benzenesulfonic acid 4-[4-methyl-3-(4-pyridin-3-yl-thiazol-2-ylamino) -phenylcarbamoyl]-phenyl ester,
N-[4-Methyl-3-(4-pyridin-3-yl-thiazol-2-ylamino)-phenyl]-4-pyrrolidin-1-ylmethyl-benzamide,
3-Methyl-N-[4-methyl-3-(4-pyridin-3-yl-thiazol-2-ylamino)-phenyl]-benzamide,
N-[4-Methyl-3-(4-pyridin-3-yl-thiazol-2-ylamino)-phenyl]-3-trifluoromethyl-benzamide,
4-[3-(2,4-Dimethoxy-phenyl)-ureido]N-[4-methyl-3-(4-pyridin-3-yl-thiazol-2-ylamino)-phenyl]-benzamide,
N-[4-Methyl-3-(4-pyridin-3-yl-thiazol-2-ylamino)-phenyl][-4-3-(4-tri fluoromethyl-phenyl)-ureidomethyl]-benzamide,
N-[4-Methyl-3-(4-pyridin-3-yl-thiazol-2-ylamino)-phenyl]-4-[3-(3,4,5-trimethoxy -phenyl)-ureido]-benzamide,
4-[3-(2-Iodo-phenyl)-ureido]-N-[4-methyl-3-(4-pyridin-3-yl-thiazol-2-ylamino) -phenyl]-benzamide,
4-[3-(4-Fluoro-phenyl)-ureido]-N-[4-methyl-3-(4-pyridin-3-yl-thiazol-2-ylamino)-phenyl]-benzamide,
2-Fluoro-benzenesulfonic acid 4-[4-methyl-3-(4-pyridin-3-yl-thiazol-2-ylamino) -phenylcarbamoyl]-phenyl ester,
3-Fluoro-benzenesulfonic acid 4-[4-methyl-3-(4-pyridin-3-yl-thiazol-2-ylamino) -phenylcarbamoyl]-phenyl ester,
2-(2-methyl-5-tert-butoxycarbonylamino)phenyl-4-(3-pyridyl)-thiazole,
2-(2-methyl-5-amino)phenyl-4-(3-pyridyl)-thiazole
4-(4-Methyl-piperazin-1-ylmethyl)-N-[3-(4-pyridin-3-yl-thiazol-2-ylamino) -phenyl]-benzamide
N-[4-Methyl-3-(4-phenyl-thiazol-2-ylamino)-phenyl]-4-(4-methyl-piperazin-1-ylmethyl)-benzamide
N-[3-([2,4′]Bithiazolyl-2′-ylamino)-4-methyl-phenyl]-4-(4-methyl-piperazin-1-ylmethyl)-benzamide,
4-(4-Methyl-piperazin-l-ylmethyl)-N-[4-methyl-3-(4-pyrazin-2-yl-thiazol-2-ylamino)-phenyl]-benzamide
2-[5-(3-Iodo-benzoylamino)-2-methyl-phenylamino]-thiazole-4-carboxylic acid ethyl ester
2-{2-Methyl-5-[4-(4-methyl-piperazin-1-ylmethyl)-benzoylamino]-phenylamino}thiazole-4-carboxylic acid ethyl ester
N-[4-Chloro-3-(4-pyridin-3-yl-thiazol 2-ylamino)-phenyl]-4-(4-methyl-piperazin-1-ylmethyl)-benzamide
3-Bromo-N-{3-[4-(4-chloro-phenyl)-5-methyl-thiazol-2-ylamino]-4-methylphenyl}-benzamide
{3-[4-(4-Chloro-phenyl)-5-methyl-thiazol-2-ylamino]-4-methyl-phenyl}-carbamic acid isobutyl ester
2-[5-(3-Bromo-benzoylamino)-2-methyl-phenylamino]-5-(4-chloro-phenyl)-thiazole-4-carboxylic acid ethyl ester
2-[5-(3-Bromo-benzoylamino)-2-methyl-phenylamino]-5-(4-chloro-phenyl)-thiazole-4-carboxylic acid (2-dimethylamino-ethyl)-amide
N-{3-[4-(4-Methoxy-phenyl)-thiazol-2-ylamino]-4-methyl-phenyl}-4-(4-methyl-piperazin-1-ylmethyl)-benzamide
4-(4-Methyl-piperazin-l-ylmethyl)-N- {4-methyl-3-[4-(3-trifluoromethyl -phenyl)-thiazol-2-ylamino]-phenyl }-benzamide
N-{4-Methyl-3-[4-(3-nitro-phenyl)-thiazol-2-ylamino]-phenyl}-4-(4-methyl-piperazin-1-ylmethyl)-benzamide
N-{3-[4-(2,5-Dimethyl-phenyl)-thiazol-2-ylamino]-4-methyl-phenyl}-4-(4-methyl-piperazin-1-ylmethyl)-benzamide
N-{3-[4-(4-Chloro-phenyl)-thiazol-2-ylamino]-4-methyl-phenyl}-4-(4-methyl-piperazin-1-ylmethyl)-benzamide
3-Bromo-4-methyl-N-[4-methyl-3-(4-pyridin-3-yl-thiazol-2-ylamino)-phenyl]-benzamide
4-Fluoro-N-[4-methyl-3-(4-pyridin-3-yl-thiazol-2-ylamino)-phenyl]-benzamide
3,5-Dibromo-N-[4-methyl-3-(4-pyridin-3-yl-thiazol-2-ylamino)-phenyl]-4-piperidin-1-ylmethyl-benzamide
N-{3-[4-(3-Fluoro-phenyl)-thiazol-2-ylamino]-4-methyl-phenyl}-4-(4-methyl-piperazin-1-ylmethyl)-benzamide
N-{3-[4-(3-Methoxy-phenyl)-thiazol-2-ylamino]-4-methyl-phenyl}-4-(4-methyl-piperazin-1-ylmethyl)-benzamide
N-{3-[4-(2-Fluoro-phenyl)-thiazol-2-ylamino]-4-methyl-phenyl}-4-(4-methyl-piperazin-1-ylmethyl)-benzamide
4(4-Methyl-piperazin-1-ylmethyl)-N-[4-methyl-3(4-pyridin-2-yl-thiazol-2-ylamino)-phenyl]-benzamide
4-Cyano-N-[4-methyl-3-(4-pyridin-3-yl-thiazol-2-ylamino)-phenyl]-benzamide
4-Fluoro-N-[4-methyl-3-(4-pyridin-3-yl-thiazol-2-ylamino)-phenyl]-benzamide
1-(2-Fluoro-phenyl)-3- [4-methyl-3-(4-pyridin-3-yl-thiazol-2-ylamino)-phenyl]-urea
1-(2-Chloro-phenyl)-3-[4-methyl-3-(4-pyridin-3-yl-thiazol-2-ylamino)-phenyl]-urea
1-(3-Fluoro-phenyl)-3-[4-methyl-3-(4-pyridin-3-yl-thiazol-2-ylamino)-phenyl]-urea
1-[4-Methyl-3-(4-pyridin-3-yl-thiazol-2-ylamino)-phenyl]-3-p-tolyl-urea
3-Bromo-N-[4-methyl-3-(4-pyridin-3-yl-thiazol-2-ylamino)-phenyl]-benzamide
N-[4-Methyl-3-(4-pyridin-3-yl-thiazol-2-ylamino)-phenyl]-4-(thiophene-2-sulfonylamino)-benzamide
3 -Fluoro-N-[4-methyl-3-(4-pyridin-3-yl-thiazol-2-ylamino)-phenyl]-benzamide
N-[4-Methyl-3-(4-pyridin-3-yl-thiazol-2-ylamino)-phenyl]-4-pyridin-4-yl-benzamide
4-Dimethylamino-N-[4-methyl-3-(4-pyridin-3-yl-thiazol-2-ylamino)-phenyl]-benzamide
2-Fluoro-5-methyl-N-[4-methyl-3-(4-pyridin-3-yl-thiazol-2-ylamino)-phenyl]-benzamide
4-tert-Butyl-N-[4-methyl-3-(4-pyridin-3-yl-thiazol-2-ylamino)-phenyl]-benzamide
4-Isopropoxy-N-[4-methyl-3-(4-pyridin-3-yl-thiazol-2-ylmethyl)-phenyl]-benzamide
Benzo[1,3]dioxole-5-carboxylic acid [4-methyl-3-(4-pyridin-3-yl-thiazol-2-ylmethyl)-phenyl]-amide
N-[4-Methyl-3-(4-pyridin-3-yl-thiazol-2-ylamino)-phenyl]-3-(2-morpholin-4-ylethoxy)-benzamide
N-[4-Methyl-3-(4-pyridin-3-yl-thiazol-2-ylmethyl)-phenyl]-4-pyridin-4-ylbenzamide
3-Cyano-N-[4-methyl-3-(4-pyridin-3-yl-thiazol-2-ylamino)-phenyl]-benzamide
2-Fluoro-N-[4-methyl-3-(4-pyridin-3-yl-thiazol-2-ylamino)-phenyl]-3-trifluoromethyl-benzamide
4-Aminomethyl-N-[4-methyl-3-(4-pyridin-3-yl-thiazol-2-ylamino)-phenyl]-benzainide
3-Methoxy-N-[4-methyl-3-(4-pyridin-3-yl-thiazol-2-ylmethyl)-phenyl]-benzamide
4-(4-Methyl-piperazin-l-yl)-N-[4-methyl-3-(4-pyridin-3-yl-thiazol-2-ylmethyl)-phenyl]-benzamide
Biphenyl-3-carboxylic acid [4-methyl-3-(4-pyridin-3-yl-thiazol-2-ylamino)-phenyl]-amide
N-[4-Methyl-3-(4-pyridin-3-yl-thiazol-2-ylamino)-phenyl]-isonicotinamide
2,6-Dichloro-N-[4-methyl-3-(4-pyridin-3-yl-thiazol-2-ylamino)-phenyl]-isonicotinamide
3,5-Dibromo-4-(4-methyl-piperazin-1-ylmethyl)-N-[4-methyl-3-(4-pyridin-3-ylthiazol-2-ylamino)-phenyl]-benzamide
3-Fluoro-4-(4-methyl-piperazin-1-ylmethyl)-N-[4-methyl-3-(4-pyridin-3-ylthiazol-2-ylamino)-phenyl]-benzamide
4-(4-Methyl-piperazin-1-ylmethyl)-N-[4-methyl-3-(4-pyridin-3-yl-thiazol-2-ylmethyl)-phenyl]-3-trifluoromethyl-benzamide
2,3,5,6-Tetrafluoro-4-(4-methyl-piperazin-1-ylmethyl)-N-[4-methyl-3-(4-pyridin-3 -yl-thiazol-2-ylamino)-phenyl]-benzamide
N-{3-[4-(4-Fluoro-phenyl)-thiazol-2-yl amino]-4-methyl-phenyl}-4-(4-methyl-piperazin-l-ylmethyl)-benzamide
3-Bromo-4-(4-methyl-piperazin-1-ylmethyl)-N-[4-methyl-3-(4-pyridin-3-ylthiazol-2-ylamino)-phenyl]-benzamide
3-Chloro-4-(4-methyl-piperazin-1-ylmethyl)-N-[4-methyl-3-(4-pyridin-3-ylthiazol-2-ylamino)-phenyl]-benzamide
4-(4-Methyl-piperazin-l-ylmethyl)-N-[4-methyl-3-(4-pyridin-4-yl-thiazol-2-ylamino)-phenyl]-benzamide
N-{3-[4-(4-Cyano-phenyl)-thiazol-2-yl amino]-4-methyl-phenyl}-4-(4-methylpiperazin-1-ylmethyl)-benzamide
4-[1-(4-Methyl-pipeazin-1-yl)-ethyl]-N-[4-methyl-3-4-pyridin-3-yl-thiazol-2-ylmethyl)-phenyl]-benzamide
4-(1-Methoxy-ethyl)-N-[4-methyl-3-(4-pyridin-3-yl-thiazol-2-ylmethyl)-phenyl]-benzamide
N-{4-Methyl-3-[4-(5-methyl-pyridin-3-yl )-thiazol-2-ylamino]-phenyl }-4-(4-methyl-piperazin-1-ylmethyl)-benzamide
3-Iodo-4-(4-methyl-piperazin-1-ylmethyl)-N-[4-methyl-3-(4-pyridin-3-yl-thiazol-2-ylmethyl)-phenyl]-benzamide
3,5-Dibromo-N-[4-methyl-3-(4-pyridin-3-yl-thiazol-2-ylamino)-phenyl]-4-[(3 morpholin-4-yl-propylamino)-methyl]-benzamide
3-Dimethylamino-N-[4-methyl-3-(4-pyridin-3-yl-thiazol-2-ylamino)-phenyl]-benzamide
3-(4-Methyl-piperazin-1-yl)-N-[4-methyl-3-(4-pyridin-3-yl-thiazol-2-ylamino)-phenyl]-benzamide
N-[4-Methyl-3-(4-pyridin-3-yl-thiazol-2-ylamino)-phenyl]-3-morpholin-4-ylbenzamide
Cyclohexanecarboxylic acid [4-methyl-3-(4-pyridin-3-yl-thiazol-2-ylmethyl)-phenyl]-amide
5-[4-Methyl-3-(4-pyridin-3-yl-thiazol-2-ylamino)-phenylcarbamoyl]-pentanoic acid ethyl ester
1-Methyl-cyclohexanecarboxylic acid[4-methyl-3-(4-pyridin-3-yl-thiazol-2-ylmethyl)-phenyl]-amide
4-tert-Butyl-cyclohexanecarboxylic acid[4-methyl-3-(4-pyridin-3-yl-thiazol-2-ylamino)-phenyl]-amide
N-[4-Methyl-3-(4-pyridin-3-yl-thiazol-2-ylamino)-phenyl]-4-morpholin-4-ylbutyramide
[4-Methyl-3-(4-pyridin-3-yl-thiazol-2-ylamino)-phenyl]-carbamic acid isobutyl ester
2-(2-methyl-5-tert-butoxycarbonylamino)phenyl-4-(3-pyridyl)-thiazole

14. A method for treating type II diabetes, obesity and related disorders comprising administering to a human in need of such treatment a compound that is a selective, potent and non toxic inhibitor of activated c-kit obtainable by a screening method which comprises:
a) bringing into contact (i) activated c-kit and (ii) at least one compound to be tested; under conditions allowing the components (i) and (ii) to form a complex,
b) selecting compounds that inhibit activated c-kit,
c) testing and selecting a subset of compounds identified in step b), which are unable to promote death dependent cells cultured in presence of IL-3.

15. A method according to claim 14, wherein the screening method further comprises the step consisting of testing and selecting a subset of compounds identified in step b) that are inhibitors of mutant activated c-kit, which are also capable of inhibiting SCF-activated c-kit wild.

16. A method according to claim 14, wherein activated c-kit is SCF-activated c-kit wild in step a).

17. A method according to claim 14, wherein putative inhibitors are tested at a concentration above 10 μM in step a).

18. A method according to claim 14, wherein IL-3 is preferably present in the culture media of dependent cells at a concentration comprised between 0.5 and 10 ng/ml, preferably between 1 to 5 ng/ml.

19. A method according to claim 14, wherein IL-3 dependent cells are selected from the group consisting of mast cells, transfected mast cells, BaF3 and IC-2.

20. A method according to claim 14, wherein the extent to which component (ii) inhibits activated c-kit is measured in vitro or in vivo.

21. A method according to claim 14, further comprising the step consisting of testing and selecting compounds capable of inhibiting c-kit wild at concentration below 1 μM.

22. A method according to claim 17, wherein the testing is performed in vitro or in vivo.

23. A method according to claim 14, wherein the inhibition of mutant- activated c-kit and/or c-kit wild is measured using standard biochemical techniques such as immunoprecipitation and western blot.

24. A method according to claim 14, wherein the amount of c-kit phosphorylation is measured.

25. A method according to claim 14, wherein identified and selected compounds are potent, selective and non-toxic c-kit wild inhibitors.

26. A method for treating type II diabetes, obesity and related disorders comprising administering to a human in need of such treatment a c-kit inhibitor obtainable by a screening method comprising:
a) performing a proliferation assay with cells expressing a mutant c-kit (for example in the transphosphorylase domain), which mutant is a permanent activated c-kit, with a plurality of test compounds to identify a subset of candidate compounds targeting activated c-kit, each having an IC50<10 μM by measuring the extent of cell death,
b) performing a proliferation assay with cells expressing c-kit wild said subset of candidate compounds identified in step (a), said cells being IL-3 dependent cells cultured in presence of IL-3, to identify a subset of candidate compounds targeting specifically c- kit,
c) performing a proliferation assay with cells expressing c-kit, with the subset of compounds identified in step b) and selecting a subset of candidate compounds targeting c-kit wild, each having an IC50<10 μM, preferably an IC50 <1 μM, by measuring the extent of cell death.

27. A method according to claim 26, wherein the extent of cell death is measured by 3H thymidine incorporation, the trypan blue exclusion method or flow cytometry with propidium iodide.

28. A method according claim 1 for preventing, delaying the onset treating type II diabetes and obesity in human.

29. A method according to claim 1 for preventing, delaying the onset and/or treating of hypercholesterolemia, hypergycemia, hypertension, endothelial dysfunction, insulin resistance, and vascular remodelling.

30. (canceled)

31. A composition suitable for oral administration comprising a compound capable of depleting mast cells, preferably a tyrosine kinase inhibitor, more particularly a c-kit inhibitor for treating for preventing, delaying the onset and/or treating type II diabetes and obesity including hypercholesterolemia, hypergycemia, hypertension, endothelial dysfunction, insulin resistance, and vascular remodelling.

32. A composition according to claim 31 suitable for intravenous, intramuscular, intraarterial, intramedullary, intrathecal, intraventricular, transdermal, subcutaneous, intraperitoneal, enteral, sublingual, or rectal administration.


SEQUENCE LISTING (5 sequences)

SEQ ID NO: 1 - protein, 976 aa, Homo sapiens [sequence omitted]

SEQ ID NO: 2 - DNA, 30 bp, Homo sapiens
  Primer
aagaagagatggtacctcgaggggtgaccc

SEQ ID NO: 3 - DNA, 33 bp, Homo sapiens
  Primer
ctgcttcgcggccgcgttaactcttctcaacca

SEQ ID NO: 4 - DNA, 20 bp, Homo sapiens
  Primer
agctcgtttagtgaaccgtc

SEQ ID NO: 5 - DNA, 20 bp, Homo sapiens
  Primer
gtcagacaaaatgatgcaac